(19) United States
(12) Patent Application Publication
(10) Pub. No.: US 2026/0020920 A1
(43) Pub. Date: Jan. 22, 2026

(54) METHODS AND SYSTEMS FOR VASCULAR TRACKING

(71) Applicant: SHANGHAI UNITED IMAGING HEALTHCARE CO., LTD., Shanghai (CN)

(72) Inventors: Keke YANG, Shanghai (CN); Tuoyu CAO, Shanghai (CN); Yanqi HUANG, Shanghai (CN); Zheng ZHANG, Shanghai (CN)

(73) Assignee: SHANGHAI UNITED IMAGING HEALTHCARE CO., LTD., Shanghai (CN)

(21) Appl. No.: 19/340,995

(22) Filed: Sep. 26, 2025

Related U.S. Application Data
(63) Continuation of application No. PCT/CN2024/106053, filed on Jul. 17, 2024.

(30) Foreign Application Priority Data
Jul. 17, 2023 (CN) 202310878414.3

(51) Int. Cl. A61B 34/20; A61B 5/00; A61B 34/10; G06T 7/00; G06T 7/246

(52) U.S. Cl. CPC A61B 34/20; A61B 5/489; A61B 34/10; G06T 7/0014; G06T 7/248; A61B 2034/2065; G06T 2207/10016; G06T 2207/20081; G06T 2207/30101

(57) ABSTRACT

Embodiments of the present disclosure provide a method and a system for vascular tracking. The method includes determining a plurality of first vascular movements and a plurality of first background movements of an object based on a reference image and a plurality of contrasted images of the object; determining structural parameters of a movement determination model based on the plurality of first vascular movements and the plurality of first background movements; and determining a predictive vascular region in a target image based on the movement determination model.

### CROSS-REFERENCE TO RELATED APPLICATIONS

This application is a continuation of International Application No. PCT/CN2024/106053, filed on Jul. 17, 2024, which claims priority to Chinese Patent Application No. 202310878414.3, filed on Jul. 17, 2023, the entire contents of which are incorporated herein by reference.

### TECHNICAL FIELD

The present disclosure relates to the field of medical imaging technology, and in particular, to methods and systems for vascular tracking.

### BACKGROUND

In a vascular interventional surgery, physicians obtain a real time position of a surgical instrument within vessels by taking X-ray images after injecting a contrast agent into the patient. The location of plaques or lesions relative to the surgical instrument is determined to facilitate subsequent surgical decision-making. However, the contrast agent flows fast and is highly toxic, resulting in that prolonged visualization of the blood vessels during the procedure cannot be provided. In addition, factors such as respiration or heartbeat cause movement of the patient's blood vessels, complicating the interventional surgery.

Therefore, it is desirable to provide methods and systems for vascular tracking, which may accurately predict the location of the vessels in a medical image after the contrast agent dissipated, thereby reducing the difficulty of the interventional surgery and decreasing the amount of contrast agent required.

### SUMMARY

One or more embodiments of the present disclosure provide a method for vascular tracking. The method comprises determining a plurality of first vascular movements and a plurality of first background movements of an object based on a reference image and a plurality of contrasted images of the object, the reference image and the plurality of contrasted images being images of blood vessels within the object which provides enhanced visualization at a region of interest, the plurality of first vascular movements representing vascular movements in the plurality of contrasted images relative to the reference image, and the plurality of first background movements represent background movements in the plurality of contrasted images relative to the reference image; determining of a movement determination model based on the plurality of first vascular movements and the plurality of first background movements, the movement determination model characterizes a vascular motion of a vascular region between at least two frames of the plurality of contrasted images, a background motion of a background region between the at least two frames of the plurality of contrasted images, or a relationship between the vascular motion and the background motion.

In some embodiments, the plurality of contrasted images include at least one of an X-ray image, a computed tomography (CT) image, or a magnetic resonance imaging (MRI) image.

In some embodiments, the determining the plurality of first vascular movements based on the reference image and the plurality of contrasted images of the object comprises determining a first vascular region in the reference image and a plurality of second vascular regions in the plurality of contrasted images; and determining the plurality of first vascular movements based on the first vascular region and the plurality of second vascular regions.

In some embodiments, the determining the plurality of first background movements based on the reference image and the plurality of contrasted images of the object comprises determining a first background region in the reference image and a plurality of second background regions in the plurality of contrasted images; and determining the plurality of first background movements based on the first background region and the plurality of second background regions.

In some embodiments, the determining the plurality of first vascular movements based on the reference image and the plurality of contrasted images of the object comprises determining the first vascular region in the reference image by processing the reference image; and determining the plurality of first vascular movements based on the first vascular region and the plurality of contrasted images.

In some embodiments, the determining the plurality of first background movements based on the reference image and the plurality of contrasted images of the object comprises determining the first background region in the reference image by processing the reference image; and determining the plurality of first background movements based on the first background region and the plurality of contrasted images.

In some embodiments, the determining the movement determination model based on the plurality of first vascular movements and the plurality of first background movements comprises determining, based on the plurality of first vascular movements, the plurality of first background movements, and a predetermined relational model, structural parameters of the movement determination model.

In some embodiments, the determining the predictive vascular region in the target image based on the movement determination model comprises determining a second background movement based on the target image and the first background region of the reference image; determining a second vascular movement based on the second background movement and the movement determination model; and determining the predictive vascular region based on the first vascular region and the second vascular movement.

In some embodiments, the movement determination model is a trained machine learning model, and the determining the movement determination model based on the plurality of first vascular movements and the plurality of first background movements comprises obtaining a plurality of training samples and a plurality of labels. The plurality of training samples include the plurality of first background movements, and the plurality of labels corresponding to the plurality of training samples include the plurality of first vascular movements. The determining the movement determination model based on the plurality of first vascular movements and the plurality of first background movements also comprises training an initial movement determination model based on the plurality of training samples and the plurality of labels; and obtaining the structural parameters of the movement determination model until a trained movement determination model satisfies a predetermined condition.

In some embodiments, there are a plurality of target images, and the method further comprises extracting the plurality of target images to form an image sequence based on a target video; and determining the predictive vascular region based on the image sequence.

In some embodiments, the determining the movement determination model based on the plurality of first vascular movements and the plurality of first background movements comprises obtaining a reliability by performing a time-domain filtering on the plurality of first vascular movements, the reliability denoting a reliability degree of the movement of a vascular point in one of the plurality of contrasted images; and determining the structural parameters of the movement determination model based on the plurality of first vascular movements, the plurality of first background movements, and the reliability.

In some embodiments, the determining the first background region in the reference image and the plurality of second background regions in the plurality of contrasted images based on the reference image and the plurality of contrasted images through the segmentation algorithm respectively comprises: obtaining a second reference image and a plurality of second contrasted images, the second reference image being a processed reference image and each of the second contrasted images being a processed contrasted image; and determining the first background region and the plurality of second background regions based on the second reference image and the plurality of second contrasted images.

In some embodiments, there is at least one target image extracted based on a target video, and the method further comprises determining at least one predictive vascular region of the at least one target image and the movement determination model, the movement determination model being a trained machine learning model.

In some embodiments, the determining the movement determination model based on the plurality of first vascular movements and the plurality of first background movements comprises determining the structural parameters of the movement determination model based on the plurality of first vascular movements, the plurality of first background movements, and a structural parameter prediction model, the structural parameter prediction model being a trained machine learning model.

In some embodiments, the determining the movement determination model based on the plurality of first vascular movements and the plurality of first background movements comprises: obtaining a vascular composite movement feature based on the plurality of first vascular movements; obtaining a background composite movement feature based on the plurality of first background movements; and determining structural parameters of the movement determination model based on the vascular composite movement feature and background composite movement feature.

One or more embodiments of the present disclosure provide a method for surgical path planning. The method comprises obtaining a plurality of contrasted images and a non-contrasted image of an object; determining a plurality of first vascular movements and a plurality of first background movements based on the plurality of contrasted images, wherein the plurality of contrasted images include a reference image, the plurality of first vascular movements represent vascular movements of the plurality of contrasted images relative to the reference image, and the plurality of first background movements represent background movements of the plurality of contrasted images relative to the reference image; determining a movement determination model, wherein the movement determination model characterizes a vascular motion of a vascular region between at least two frames of the plurality of contrasted images a background motion of a background region between the at least two frames of the plurality of contrasted images, or a relationship between the vascular motion and the background motion; providing enhanced visualization of a predictive vascular region in the non-contrasted image; determining a predictive vascular region in a target image based on the method for vascular tracking; and generating a planning path for a vascular interventional surgery based on the predictive vascular region in the non-contrasted image.

One or more embodiments of the present disclosure provide a system for vascular tracking. The system comprises a first determination module, a second determination module, and a third determination module. The first determination module is configured to determine a plurality of first vascular movements and a plurality of first background movements of an object based on a reference image and a plurality of contrasted images of the object. Both the reference image and the plurality of contrasted images are images of blood vessels within the object which provides enhanced visualization at a region of interest, the plurality of first vascular movements represent vascular movements in the plurality of contrasted images relative to the reference image, and the plurality of first background movements represent background movements in the plurality of contrasted images relative to the reference image. The second determination module is configured to determine a movement determination model based on the plurality of first vascular movements and the plurality of first background movements. The movement determination model characterizes a vascular motion of a vascular region between at least two frames of the plurality of contrasted images, and a background motion of a background region between the at least two frames of the plurality of contrasted images, or a relationship between the vascular motion and the background motion. The third determination module is configured to determine a predictive vascular region in a target image based on the movement determination model, the target image being a non-contrasted image of the object.

One or more embodiments of the present disclosure provide a system for surgical path planning. The system comprises a first acquiring module, a first determination module, a second determination module, a third determination module, and a generating module. The first acquiring module is configured to obtain a plurality of contrasted images and a non-contrasted image of an object. The first determination module is configured to determine a plurality of first vascular movements and a plurality of first background movements based on the plurality of contrasted images. The plurality of contrasted images include a reference image, the plurality of first vascular movements represent vascular movements of the plurality of contrasted images relative to the reference image, and the plurality of first background movements represent background movements of the plurality of contrasted images relative to the reference image. The second determination module is configured to determine a movement determination model. The movement determination model characterizes a motion matrix of a vascular region between at least two frames of the plurality of contrasted images. The third determination module is configured to determine a predictive vascular region in the non-contrasted image. The second acquiring module is configured to obtain a relative position of a surgical instrument in the predictive vascular region based on position information of the predictive vascular region. The generating module is configured to generate a planning path for a vascular interventional surgery based on the relative position and a preoperative path.

One or more embodiments of the present disclosure provide a method for vascular tracking. The method includes obtaining a plurality of contrasted images of an object, each of the plurality of contrasted images including a vascular region and a background region; determining a movement determination model based on a plurality of vascular regions and background regions of the plurality of contrasted images; and determining a predictive vascular region in a non-contrasted image of the object based on the movement determination model, The movement determination model characterizes a vascular region movement between different contrasted images, a background region movement between different contrasted images, or a relationship between the vascular region movement and the background region movement.

One or more embodiments of the present disclosure provide a system for vascular tracking including a second acquiring module, a fourth determination module and a fifth determination module. The second acquiring module is configured to obtain a plurality of contrasted images of an object, each of the plurality of contrasted images including a vascular region and a background region. The fourth determination module is configured to determine a movement determination model based on a plurality of vascular regions and background regions of the plurality of contrasted images. The movement determination model characterizes a vascular region movement between different contrasted images, a background region movement between different contrasted images, or a relationship between the vascular region movement and the background region movement. The fifth determination module is configured to determine a predictive vascular region in a non-contrasted image of the object based on the movement determination model.

One or more embodiments of the present disclosure provide a method for vascular tracking. The method comprises obtaining a movement determination model; obtaining a non-contrasted image of a latter object; and determining a predictive vascular region in the non-contrasted image of the latter object based on the movement determination model. The movement determination model represents a vascular region movement between different contrasted images, a background region movement between different contrasted images, or a relationship between the vascular region movement and the background region movement, of a prior object.

One or more embodiments of the present disclosure provide a method for vascular tracking. The method comprises obtaining a non-contrasted image of an object; obtaining a movement determination model; and determining a predictive vascular region in the non-contrasted image of the object based on the movement determination model. The movement determination model is able to predict a vascular region in a target image of the object without needing of the phase information of the object.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a schematic diagram illustrating an exemplary system for vascular tracking according to some embodiments of the present disclosure;

FIG. 2 is a diagram illustrating modules of an exemplary system for vascular tracking according to some embodiments of the present disclosure;

FIG. 3 is a schematic diagram illustrating an exemplary method for vascular tracking according to some embodiments of the present disclosure;

FIG. 4 is a schematic diagram illustrating a contrasted image according to some embodiments of the present disclosure;

FIG. 5 is a schematic diagram illustrating a target image in a vascular region according to some embodiments of the present disclosure;

FIG. 6 is a schematic diagram illustrating a vascular respiratory motion compensation according to some embodiments of the present disclosure;

FIG. 7 is a schematic diagram illustrating an exemplary method for vascular tracking according to some embodiments of the present disclosure;

FIG. 8 is an exemplary flowchart illustrating an exemplary method for surgical path planning according to some embodiments of the present disclosure;

FIG. 9 is a diagram illustrating modules of an exemplary system for surgical path planning according to some embodiments of the present disclosure; and

FIG. 10 is a schematic diagram illustrating an exemplary electronic device for vascular tracking in a medical image according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system,” “device,” “unit,” and/or “module” as used herein is a manner used to distinguish different components, elements, parts, sections, or assemblies at different levels. However, if other words serve the same purpose, the words may be replaced by other expressions.

As shown in the present disclosure and claims, the words “once,” “a,” “a kind,” and/or “the” are not especially singular but may include the plural unless the context expressly suggests otherwise. In general, the terms “comprise,” “comprises,” “comprising,” “include,” “includes,” and/or “including”, merely prompt to include operations and elements that have been clearly identified, and these operations and elements do not constitute an exclusive listing. The methods or devices may also include other operations or elements.

Flowcharts are used in the present disclosure to illustrate operations performed by systems according to embodiments of the present disclosure. It should be appreciated that the preceding or following operations are not necessarily performed in an exact sequence. Instead, steps may be processed in reverse order or simultaneously. Also, it is possible to add other operations to these processes or remove a step or steps from them.

To ensure a system for vascular tracking can continue to predict the location of vascular regions after the disappearance of a contrast agent, thereby effectively assisting physicians during surgery, shortening surgery time, and improving the accuracy of the procedure while reducing the amount of contrast agent required, a method of vascular respiratory motion compensation is mainly employed.

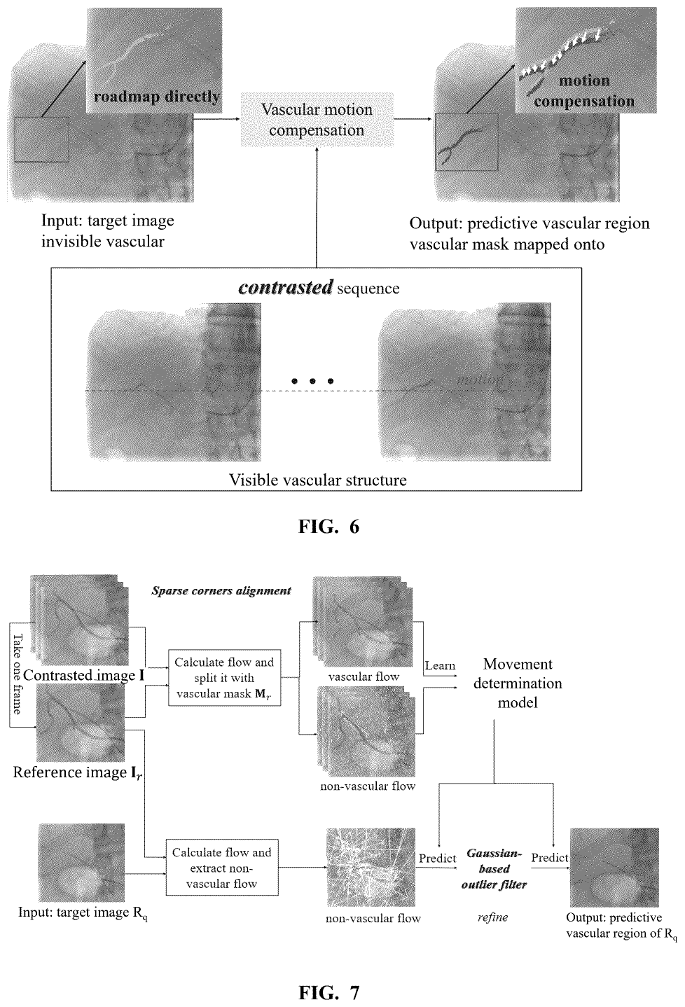

FIG. 6 is a schematic diagram illustrating vascular respiratory motion compensation according to some embodiments of the present disclosure. As shown in FIG. 6, a target image with invisible vascular structures is input into a vascular respiratory motion compensation system, and the vascular respiratory motion compensation system outputs a predictive vascular region with a mapped dark vascular mask. The predictive vascular region is obtained by mapping contrasted sequences onto the target image directly, as indicated by a light vascular mask. The vascular respiratory motion compensation system utilizes contrasted sequences to predict motion between the target image and the predictive vascular region, as denoted by white arrows in FIG. 6. The dotted line is used for easy visualization of motion.

Existing vascular respiratory motion compensation methods may be categorized into respiratory state-driven methods and motion modeling methods. Although the respiratory state-driven methods are time-efficient, their accuracy and robustness are limited because motions over a respiratory cycle are generally unreproducible. On the other hand, the motion modeling methods may yield high accuracy and robust compensations without additional input, but they require significant computational resources. It is required to achieve both real-time implementation and high accuracy in the vascular respiratory motion compensation.

In some embodiments of the present disclosure, in order to solve the problem of accurate prediction of the location of blood vessels in a medical image by a system for vascular tracking after the disappearance of the contrast agent, in the present disclosure, structural parameters of a movement determination model are determined based on a plurality of contrasted images and a reference image, to determine a predictive vascular region in a target image based on the movement determination model. Thus, the location of the blood vessels may still be predicted after the disappearance of the contrast agent, which may effectively assist the physician in the surgery, shorten the surgery time, improve the accuracy of the surgery, and thus may reduce the amount of the contrast agent required.

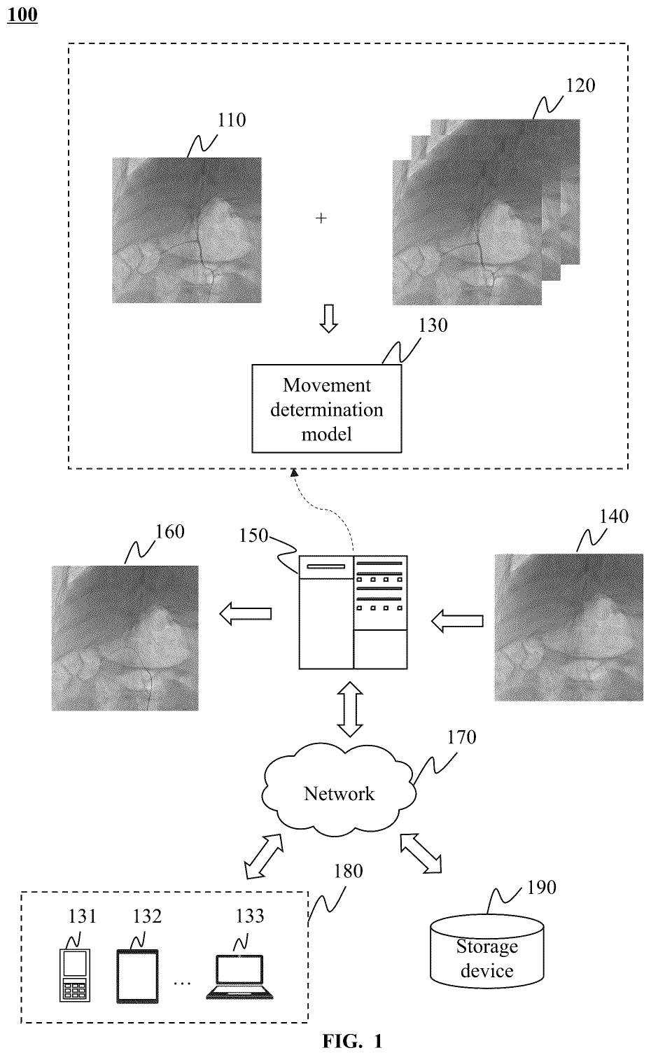

FIG. 1 is a schematic diagram illustrating an exemplary system for vascular tracking according to some embodiments of the present disclosure. In some embodiments, as shown in FIG. 1, an application scenario 100 of the system for vascular tracking may include a reference image 110, a contrasted image 120, a movement determination model 130, a target image 140, a processing device 150, a target image in which a vascular region is displayed 160, a network 170, a terminal device 180, and a storage device 190.

The contrasted image 120 refers to a contrasted image which provides enhanced visualization at a region of interest. For example, as shown in FIG. 1, the contrasted image 120 is a contrasted image of the liver.

The reference image 110 is an image that is used as a reference in the contrasted image. For example, the dark lines in the reference image 110 and the contrasted image 120 represent all the blood vessels that are visualized by the contrast agent, as shown in FIG. 1.

The movement determination model 130 refers to a model configured to identify a predictive vascular region in a non-contrasted image when there is no contrast agent present at the region of interest.

The target image 140 refers to a medical image after all or part of the contrast agent disappeared. For example, all of the blood vessels within the liver of the target image 140 are not visualized by the contrast agent, as shown in FIG. 1.

The target image in which the vascular region is displayed 160 refers to a region in the target image 140 where the blood vessel is located. The target image in which the vascular region is displayed 160 is predicted based on the movement determination model 130. For example, the dark lines in the target image in which the vascular region is displayed 160 represent the vascular region which is predicted, as shown in FIG. 1.

The processing device 150 may be configured to process data related to the vascular tracking. For example, the processing device 150 may determine a plurality of first vascular movements and a plurality of first background movements of the object based on the reference image 110 and a plurality of contrasted images 120 of the object. The processing device 150 may determine structural parameters of the movement determination model 130 based on the plurality of first vascular movements and the plurality of first background movements. The processing device 150 may determine the target image in which the vascular region is displayed 160 in the target image 140 based on the movement determination model 130.

In some embodiments, the processing device 150 may be a single server or a group of servers. The group of servers may be centralized or distributed. In some embodiments, the processing device 150 may be local or remote. In some embodiments, the processing device 150 may be implemented on a cloud platform. Merely by way of example, the cloud platform may include a private cloud, a public cloud, a hybrid cloud, a community cloud, a distributed cloud, an on-premises cloud, a multi-tier cloud, etc., or any combination thereof.

The network 170 may include any suitable network that may facilitate the exchange of information and/or data for vascular tracking. In some embodiments, one or more components of the system for vascular tracking (e.g., the processing device 150, the terminal device 180, the storage device 190) may communicate information and/or data with one or more other components of the system for vascular tracking via the network 170. For example, the processing device 150 may obtain one or more instructions from the terminal device 180 via the network 170. As another example, the processing device 150 may obtain the reference image 110, the contrasted image 120, the target image 140, or the like, from the storage device 190 via the network 170.

The terminal device 180 refers to one or more terminal devices or software used by a user. The terminal device 180 may include a mobile device, a tablet, a laptop, or the like, or any combination thereof. In some embodiments, the processing device 150 may present the target image in which the vascular region is displayed 160 to the user via the terminal device 180, facilitating the physician to be able to, make an accurate prediction after the disappearance of the contrast agent.

The storage device 190 may store data, instructions, and/or any other information. In some embodiments, the storage device 190 may store data and/or instructions related to the system for vascular tracking. For example, the storage device may store the structural parameters of the movement determination model. As another example, the storage device may store instructions for determining the predictive vascular region in the target image.

In some embodiments, the storage device 190 may include a mass storage device, a removable storage device, a volatile read-write memory, a read-only memory (ROM), or the like, or any combination thereof.

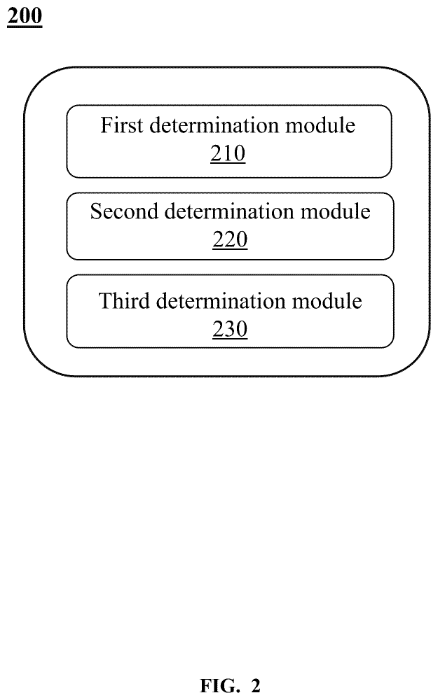

More descriptions regarding parameters of the reference image 110, the contrasted image 120, the movement determination model 130, the target image 140, the target image in which the vascular region is displayed 160, the first vascular movements, the first background movements, etc., may be found in FIG. 2-FIG. 10 and the descriptions thereof.

It should be noted that the application scenario 100 of the system for vascular tracking is provided for illustrative purposes only and is not intended to limit the scope of the present disclosure. For those of ordinary skill in the art, a plurality of modifications or variations may be made according to the description of the present disclosure. For example, the application scenario 100 of the system for vascular tracking may be implemented on other devices to achieve similar or different functions. However, such modifications or variations do not depart from the scope of the present disclosure.

FIG. 2 is a diagram illustrating modules of an exemplary system for vascular tracking according to some embodiments of the present disclosure.

In some embodiments, the modules of the system for vascular tracking 200 may include a first determination module 210, a second determination module 220, and a third determination module 230.

The first determination module 210 may be configured to determine a plurality of first vascular movements based on a reference image and a plurality of contrasted images of an object.

In some embodiments, the first determination module 210 may be further configured to determine a first vascular region in the reference image and a plurality of second vascular regions in the plurality of contrasted images. The first determination module 210 may be further configured to determine the plurality of first vascular movements based on the first vascular region and the plurality of second vascular regions.

In some embodiments, the first determination module 210 may be further configured to determine the first vascular region in the reference image by processing the reference image. The first determination module 210 may be further configured to determine the plurality of first vascular movements based on the first vascular region and the plurality of contrasted images.

In some embodiments, the first determination module 210 may be further configured to obtain the first reference image and the plurality of first contrasted images by performing a noise filtering on the reference image and the plurality of contrasted images at least once. The first reference image is a noise filtered reference image. Each of the plurality of first contrasted image is a noise-filtered contrasted image. The noise filtering includes at least one of a Gaussian filtering or a Hessian filtering. The first determination module 210 may determine the first vascular region and the plurality of second vascular regions based on the first reference image and the plurality of first contrasted images through the segmentation algorithm respectively. For example, the first determination module 210 may determine the first vascular region based on the first reference image through the segmentation algorithm. The first determination module 210 may determine the plurality of second vascular regions based on the plurality of first contrasted images through the segmentation algorithm.

In some embodiments, the first determination module 210 may be further configured to obtain a second reference image and a plurality of second contrasted images. The second reference image is a processed reference image. Each second contrasted image is a processed contrasted image. The first determination module 210 may determine the plurality of second background regions based on the plurality of second contrasted images.

The first determination module 210 may be configured to determine a plurality of first background movements based on the reference image and the plurality of contrasted images of the object.

In some embodiments, the first determination module 210 may be further configured to y determine the first background region in the reference image and the plurality of second background regions in the plurality of contrasted images. The first determination module 210 may determine the plurality of first background movements based on the first background region and the plurality of second background regions.

In some embodiments, the first determination module 210 may be further configured to determine the first background region in the reference image by processing the reference image. The first determination module 210 may be further configured to determine the plurality of first background movements based on the first background region and the plurality of contrasted images.

In some embodiments, the reference image and the plurality of contrasted images refer to images of blood vessels within the object which provides enhanced visualization at a region of interest, the first vascular movement refers to a movement of vessels in the contrasted image with respect to the reference image, and the first background movement refers to a movement of backgrounds in the contrasted image with respect to the reference image.

In some embodiments, the plurality of contrasted images may include an X-ray image, a computed tomography (CT) image, a magnetic resonance imaging (MRI) image, or the like, or any combination thereof.

The second determination module 220 may be configured to determine structural parameters of a movement determination model based on the plurality of first vascular movements and the plurality of first background movements.

In some embodiments, the movement determination model may characterize a vascular motion of a vascular region between at least two frames of the plurality of contrasted images, a background motion of a background region between the at least two frames of the plurality of contrasted images, or a relationship between the vascular motion and the background motion.

In some embodiments, the second determination module 220 may be further configured to determine the structural parameters of the movement determination model based on the plurality of first vascular movements, the plurality of first background movements, and a predetermined relational model.

In some embodiments, the movement determination model is a machine learning model, and the second determination module 220 may be further configured to obtain a plurality of training samples and a plurality of labels. The plurality of training samples may include the plurality of first background movements, and the plurality of labels label corresponding to the plurality of training samples may include the plurality of first vascular movements. The second determination module 220 may be further configured to train an initial movement determination model based on the plurality of training samples and the plurality of labels. The second determination module 220 may be further configured to obtain the movement determination model until a trained movement determination model satisfies a predetermined condition.

In some embodiments, the second determination module 220 may be further configured to obtain a confidence level by performing a time-domain filtering on the plurality of first vascular movements. The confidence level refers to a reliability degree of a movement of a vascular point in one of the plurality of contrasted images. The second determination module 220 may be further configured to determine the structural parameters of the movement determination model based on the plurality of first vascular movements, the plurality of first background movements, and the confidence level.

In some embodiments, the second determination module 220 may be further configured to determine the structural parameters of the movement determination model based on the plurality of first vascular movements, the plurality of first background movements, and a structural parameter prediction model. The structural parameter prediction model is a trained machine learning model.

In some embodiments, the second determination module 220 may be further configured to obtain a vascular composite movement feature based on the plurality of first vascular movements. The second determination module 220 may obtain a background composite movement feature based on the plurality of first background movements. The second determination module 220 may determine the structural parameters of the movement determination model based on the vascular composite movement feature and the background composite motion feature.

The third determination module 230 may be configured to determine a predictive vascular region in the target image based on the movement determination model.

In some embodiments, the target image is a non-contrasted image of the same object.

In some embodiments, the third determination module 230 may be further configured to determine a second background movement based on the target image and the first background region in the reference image. The third determination module 230 may determine a second vascular movement based on the second background movement and the movement determination model. The third determination module 230 may determine the predictive vascular region based on the first vascular region and the second vascular movement.

In some embodiments, there are a plurality of target images, the third determination module 230 may be further configured to extract the plurality of target images to form an image sequence based on a target video. The third determination module 230 may determine the predictive vascular region based on the image sequence.

In some embodiments, the third determination module 230 may be further configured to determine at least one predictive vascular region based on the target video and the movement determination model, the movement determination model being a machine learning model.

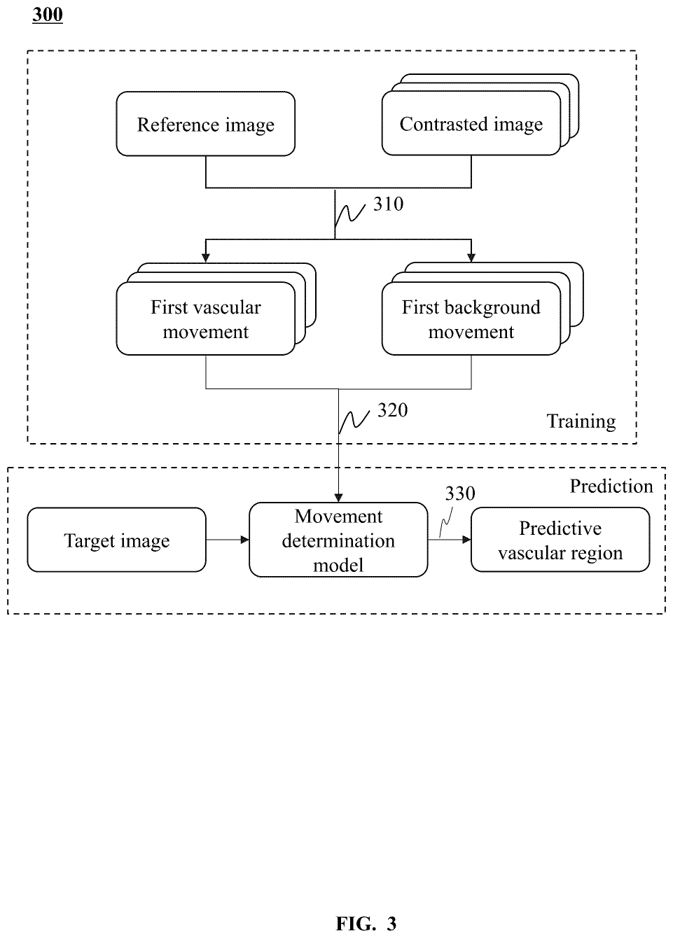

More descriptions regarding the first determination module 210, the second determination module 220, and the third determination module 230 may be found in FIG. 3-FIG. 10 and related descriptions thereof.

It is to be understood that the system and its modules shown in FIG. 2 may be realized in a plurality of ways. It should be noted that the above description of the system for vascular tracking and its modules is for descriptive convenience only and does not limit the present disclosure to the scope of the embodiments. It is to be understood that for a person skilled in the art, after understanding the principle of the system, it may be possible to arbitrarily combine each module or form a subsystem to connect with other modules without departing from this principle. In some embodiments, the first determination module 210, the second determination module 220, and the third determination module 230 disclosed in FIG. 2 may be different modules in a system, or may be a single module for implementing the functions of two or more of the above-described modules. For example, each module may share a common storage module, or may have a respective storage module. Such morphisms are within the scope of protection of the present disclosure.

FIG. 3 is a schematic diagram illustrating an exemplary method for vascular tracking according to some embodiments of the present disclosure. As shown in FIG. 3, a process 300 includes the following operations. In some embodiments, one or more of the operations of the process 300 as shown in FIG. 3 may be realized in the application scenario 100 of the system for vascular tracking shown in FIG. 1. For example, the process 300 illustrated in FIG. 3 may be stored in the form of an instruction in the storage device 190 and invoked and/or executed by the processing device 150.

In some embodiments, steps 310-320 illustrate a determination process (e.g., a training process) of the movement determination model, and step 330 is a prediction process using the movement determination model.

In 310, a plurality of first vascular movements and a plurality of first background movements are determined based on a reference image and a plurality of contrasted images of an object. In some embodiments, step 310 may be performed by the first determination module 210.

The object refers to a region of a scan subject (e.g., a patient) where a vascular surgical operation is to be performed. For example, if the scan subject requires a pulmonary vascular surgical operation, the object is the lungs.

A contrasted image refers to an image in which the blood vessels within the object are visualized by a contrast agent. In some embodiments, the contrasted image may be an image obtained by injecting or ingesting a substance containing the contrast agent into the object before performing a medical imaging technique (such as X-rays, computed tomography (CT) scans, magnetic resonance imaging (MRI), or the like) on the object.

In some embodiments, the contrasted image may include a two-dimensional image, a three-dimensional image, or the like. When the contrasted image is a two-dimensional image, each frame of the contrasted image may include a plurality of pixels. When the contrasted image is a three-dimensional image, each frame of the image may include a plurality of voxels. For ease of illustration, the following are all illustrated with the contrasted image as a two-dimensional image.

In some embodiments, the plurality of contrasted images may be obtained. One of the plurality of contrasted images may be selected as the reference image. At least a portion of the plurality of contrasted images other than the reference image may be selected as the plurality of contrasted images.

In some embodiments, the plurality of contrasted images may include a plurality of consecutive frames in the time domain. In some embodiments, the contrasted image may include a vascular region and a background region.

The vascular region refers to a specific region involving blood vessels. In the contrasted image, the vascular region is a region in which the contrast agent is developed. In some embodiments, the vascular region may include a cerebrovascular region, a cardiovascular region, a peripheral vascular region, or the like.

The background region refers to a region in the contrasted image other than blood vessels. In some embodiments, the background region may include a bone region, a soft tissue region, a visceral region, or the like.

Due to the need to determine the first vascular movements and the first background movements, in order to include a reference region, the background region may include regions (e.g., the bone region) that do not move with the heartbeat and/or respiration. The reference region refers to a region selected for reference when determining the first vascular movement and the first background movement. The reference region does not move with heartbeat and/or breathing.

In some embodiments, the processing device may obtain the contrasted image of the object of the scan subject from a medical imaging system such as a clinical information system of a hospital or a radiology information management system. The plurality of contrasted images may be a plurality of consecutive contrasted frames of one scan subject obtained by detecting the object in a historical time period, or a plurality of consecutive contrasted frames of the object of the scan subject obtained by detecting the object in a current time period. The historical time period refers to a time period corresponding to a vascular surgery operation on the same object of the same scan subject before a current vascular surgery operation is carried out. The current time period refers to a time period in which the current vascular surgery operation is carried out, and the current time period is a detection time period of the target image. The historical time period may be 3 months, 6 months, 9 months, or a longer interval from the current time period. Contrasted images in the historical time period may be registered based on the target image of a detection subject, to meet the consistency of scans in different periods and obtain intermediate historical contrasted images. By processing the intermediate historical contrasted images, the movement determination model may be obtained.

Understandably, since the heartbeat and/or respiration of the same scan subject is similar, the contrasted image of the same object of the same scan subject in the historical time period may be used, or the contrasted image of the same object of the same scan subject in the current time period may be used.

The reference image refers to an image that serves as a baseline in the contrasted images. In some embodiments, the reference image and the plurality of contrasted images refer to images of blood vessels within the object that are developed by the contrast agent.

In some embodiments, the reference image may be one frame of the plurality of contrasted images. When the plurality of contrasted images are consecutive contrasted frames of one scan subject obtained by detecting the object in the historical time period, the reference image is one frame of one scan subject obtained by detecting the object in the historical time period. When the plurality of contrasted images are consecutive contrasted frames of one scan subject obtained by detecting the object in the current time period, the reference image is one frame of one scan subject obtained by detecting the object in the current time period.

In some embodiments, the reference image is not included in the plurality of contrasted images. The reference image and the plurality of contrasted images are images of blood vessels within the object which provides enhanced visualization at the region of interest. In some embodiments, the processing device may obtain the plurality of contrasted images. The processing device may select one of the plurality of contrasted images as the reference image. The processing device may select at least a portion of the plurality of contrasted images other than the reference image as the plurality of contrasted images.

In some embodiments, the processing device may automatically obtain the reference image. For example, the processing device may designate any one of the frames in the contrasted images as the reference image. As another example, the processing device may designate a frame of the contrasted image in which a total area of blood vessels is the largest as the reference image. As yet another example, the processing device may designate a frame of the contrasted image in which a vascular segment of interest has the greatest contrast quality (e.g., the vascular segment of interest has the greatest weighted value of contrast and resolution) as the reference image. The vascular segment of interest may be a vascular segment in which the user performs a vascular surgical operation.

In some embodiments, the processing device may display a plurality of contrasted frames of the contrasted image to the user via the terminal device, and the user may manually select one of the contrasted frames as the reference image.

A first vascular movement refers to a movement of vessels in each contrasted image relative to the reference image. In some embodiments, the first vascular movement may be represented as a matrix. Each element of the matrix represents each pixel in a vascular region of a contrasted image and a movement of the each pixel point along an x-direction and/or a y-direction in the contrasted image with respect to corresponding pixel in the vascular region of the reference image. The x-direction and the y-direction may be predetermined. For example, the processing device may determine a direction perpendicular to a sagittal plane of the scan subject as the x-direction, and a direction perpendicular to a cross-section of the scan subject as the y-direction.

A first background movement refers to a movement of backgrounds in each contrasted image relative to the reference image. In some embodiments, the first background movement may be represented as a matrix. Each element of the matrix represents each pixel point in a background region of a contrasted image, and a movement of the each pixel point along the x-direction and/or the y-direction in the contrasted image with respect to corresponding pixel in the background region of the reference image. Descriptions regarding the x-direction and the y-direction may be found in the corresponding description above.

It should be noted that since there are a plurality of contrasted images and one reference image, the vascular movement of each contrasted image with respect to the reference image determines the first vascular movements, and thus, there are a plurality of first vascular movements. For similar reasons, there are a plurality of first background movements.

When the plurality of contrasted images include the reference image, the first vascular movement and the first background movement are zero matrices.

In some embodiments, the processing device may determine a first vascular region in the reference image and a plurality of second vascular regions in the plurality of contrasted images by processing the reference image and the plurality of contrasted images, respectively, based on a segmentation algorithm. The processing device may determine the plurality of first vascular movements based on pixel points corresponding to the first vascular region and pixel points corresponding to the plurality of second vascular regions through, e.g., an optical flow algorithm. In some embodiments, the processing device may determine a first background region in the reference image, and a plurality of second background regions in the plurality of contrasted images by processing the reference image and the plurality of contrasted images based on the segmentation algorithm, respectively. The processing device may determine the plurality of first background movements based on pixel points corresponding to the first background region and pixel points corresponding to the plurality of second background regions through the optical flow algorithm.

In some embodiments, the plurality of contrasted images include the reference image, and the processing device may determine the first vascular region in the reference image and the plurality of second vascular regions in the plurality of contrasted images by processing the plurality of contrasted images based on the segmentation algorithm. The processing device may determine the plurality of first vascular movements based on the pixel points corresponding to the first vascular region and the pixel points corresponding to the plurality of second vascular regions through the optical flow algorithm. In some embodiments, the plurality of contrasted images include the reference image, and the processing device may determine the first background region in the reference image and the plurality of second background regions in the plurality of contrasted images by processing the plurality of contrasted images based on the segmentation algorithm. The processing device may determine the plurality of first background movements based on the pixel points corresponding to the first background region and the pixel points corresponding to the plurality of second background regions through the optical flow algorithm.

In some embodiments, the processing device may also obtain the plurality of first vascular movements or the plurality of first background movements through other algorithms (e.g., a frame difference algorithm, a background difference algorithm etc.).

The segmentation algorithm refers to an algorithm that separates vascular regions and background regions of a medical image. For example, the segmentation algorithm may be a thresholding algorithm, an edge detection algorithm, a clustering algorithm, a deformable modeling algorithm, a region segmentation algorithm, an Otsu algorithm, a region growing algorithm, a sato filtering algorithm, a watershed algorithm, or the like. As another example, the segmentation algorithm may be a deep learning-based blood vascular segmentation algorithm, etc. Exemplarily, the deep learning-based vascular segmentation algorithm may be a vascular segmentation model.

The first vascular region refers to a region in the reference image where the blood vessels are located. In some embodiments, the first vascular region is a region in the reference image where the contrast agent is developed.

A second vascular region refers to a region in each contrasted image where the blood vessels are located. In some embodiments, the second vascular region is a region in each contrasted image where the contrast agent is developed.

The first background region refers to a region of the reference image other than the blood vessels. In some embodiments, the first background region is a region in the reference image where the contrast agent is not developed.

A second background region refers to a region of each contrasted image other than the blood vessels. In some embodiments, the second background region is a region in each contrasted image where the contrast agent is not developed.

In some embodiments, for an m×n image Iᵢ∈ [symbol] ^{m×n}, the processing device may process the image by a blood vascular segmentation algorithm, to obtain a blood vascular template Mᵢ∈ [symbol] ^{m×n}. Each pixel value in Mᵢ is denoted by 0 or 1, 0 denotes that a pixel location in the image is a background region, and I denotes that the pixel location in the image is a blood vascular region. m denotes a count of pixels in a vertical direction of the image, which is also referred to as a height of the image, and n denotes the count of pixels in a horizontal direction of the image, which is also referred to as the width of the image. Exemplarily, the processing device may determine the blood vascular template Mᵢ of the image by equation (1):

$${M}_{i} = {f}_{s} ( {I}_{i} ) , \qquad ( 1 )$$

where Mᵢ denotes the vascular template of the image, Iᵢ denotes the image, and fₛ denotes the vascular segmentation algorithm.

In some embodiments, the processing device may adopt the segmentation algorithm, such as a thresholding algorithm, an edge detection algorithm, or the like, to perform a segmentation process on the reference image to divide the first vascular region and the first background region, and perform the segmentation process on the each contrasted image to divide the second vascular region and the second background region.

In some embodiments, the processing device may input the reference image into the vascular segmentation model, and the vascular segmentation model outputs the first vascular region and the first background region. The processing device may input each contrasted image into the vascular segmentation model, and the vascular segmentation model outputs the second vascular region and the second background region. The blood vascular segmentation model may include convolutional neural networks (CNN), deep neural networks (DNN), recurrent neural networks (RNN), etc., or a combination thereof. The processing device may train the vascular segmentation model using historical contrasted images as training data, such that the vascular segmentation model may output, based on the contrasted image, the vascular region and the background region corresponding to the contrasted image. Labels corresponding to the training data may be determined by manual labeling.

In some embodiments, the processing device may process the reference image and the each contrasted image using other manners to determine the first vascular region, the first background region, the second vascular region, and the second background region. For example, the processing device may obtain the first vascular region and the plurality of second vascular regions of the plurality of contrasted images. The first vascular region and the plurality of second vascular regions may be manually outlined by a user (e.g., a physician, an operating technician, etc.) and transmit to the processing device.

In some embodiments, the processing device may perform a noise filtering on the reference image and the plurality of contrasted images at least once to obtain a first reference image and a plurality of first contrasted images. The processing device determine the first vascular region and the plurality of second vascular regions by processing the first reference image and the plurality of first contrasted images respectively based on the segmentation algorithm. The first reference image is a noise-filtered reference image, and each of the first contrasted images is a contrasted image after noise filtering. The noise filtering may include at least one of a Gauss filtering or a Hessian filtering.

By denoising the reference image and the contrasted image, a contrast between the blood vessels and the background may be improved, making it easy to segment the blood vessels, which may enhance visualization of the blood vessels and improve the correctness of the acquisition of the blood vessels, even though some of the details of the background are sacrificed.

In some embodiments, the processing device may determine a count of the noise filtering and an intensity of the noise filtering for each noise filtering based on an amount of image data and image clarity of the plurality of contrasted images. In some embodiments, the processing device may determine the count of the noise filtering and the intensity of the noise filtering for each noise filtering based on the amount of image data and image clarity of the reference image. In some embodiments, the processing device may determine the count of the noise filtering and the intensity of the noise filtering for each noise filtering based on the amount of image data and the image clarity of the plurality of contrasted images and the amount of image data and the image clarity of the reference image. The count of the noise filtering refers to a total count of the noise filtering performed on the reference image or each of the plurality of contrasted images; and the intensity of the noise filtering refers to a physical quantity reflecting the intensity of noise filtering for each noise filtering.

It should be noted that in each noise filtering, all images in the reference image and the plurality of contrasted images are noise filtered. Therefore, the count of noise filtering is increased by 1 if the noise filtering is performed once for all the images in the reference image and the plurality of contrasted images. The intensity of noise filtering performed once for each image in the reference image and the plurality of contrasted images is equal to the intensity of the current noise filtering.

The amount of image data refers to a size that an image occupies in memory. The image clarity refers to a physical quantity that used for assessing the clearness of an image. The processing device may determine an image clarity of an image based on a device that capture the image.

In some embodiments, the processing device may determine, based on the amount of image data and the image clarity of the plurality of contrasted images, the count of the noise filtering and the intensity of the noise filtering for each noise filtering by database matching. In some embodiments, the processing device may determine the count of the noise filtering and the intensity the noise filtering of each noise filtering by database matching based on the amount of image data and the image clarity of the reference image. In some embodiments, the processing device may determine the count of the noise filtering and the intensity of the noise filtering of each noise filtering by database matching based on the amount of the image data and the image clarity of the plurality of contrasted images and the amount of the image data and the image clarity of the reference image.

For example, the processing device may designate historical data in a historical database, which is subsequently determined in the historical vascular region, as sample data. Next, the processing device may designate an amount of historical contrasted image data, an amount of historical reference image data, historical image clarities, an actual count of noise filtering, and an actual intensity of noise filtering of the sample data as a clustering vector for clustering. Then, the processing device may construct reference vectors of a database using the amount of historical contrasted image data, the amount of historical reference image data, the historical image clarities of a predetermined count of clustering centers obtained by clustering, and designate the actual count of noise filtering and the actual intensity of noise filtering as labels of the reference vectors.

The processing device may construct a matched vector using an amount of the image data and an image clarity of the current contrasted image, and an amount of the image data and an image clarity of the reference image. The processing device may determine a vector similarity between the matched vector and a reference vector in the database, and determine a label corresponding to a reference vector with the highest vector similarity as the count of noise filtering and the intensity of noise filtering.

In some embodiments, the processing device may obtain, based on the reference image and the plurality of contrasted images, the second reference image and a plurality of second contrasted images through the predetermined algorithm. The processing device may determine the first background region and the plurality of second background regions by processing the second reference image and the plurality of second contrasted images, respectively, based on the segmentation algorithm. The second reference image is a processed reference image, and each of the second contrasted images is a processed contrasted image. It is to be noted that the first reference image is a noise-filtered reference image, and the second reference image is a reference image processed by the predetermined algorithm. Each of the first contrasted images is a noise-filtered contrasted image, and each of the second contrasted images is a contrasted image processed by the predetermined algorithm.

In some embodiments, the predetermined algorithm may include processing, by the processing device, the reference image and the plurality of contrasted images respectively based on the segmentation algorithm, and filling, by the processing device, each identified vascular pixel with a non-vascular pixel closest to the identified vascular pixel. Descriptions regarding the segmentation algorithm may be found in the corresponding description above.

Understandably, there may still be some vascular pixels in the second reference image and the second contrasted image, i.e., there are some vascular pixels that are not filled with the non-vascular pixels. Thus, the second reference image and the second contrasted image need to be further processed using the segmentation algorithm.

Processing the reference image and the plurality of contrasted images directly based on the segmentation algorithm may result in the presence of some blood vascular regions in the background region. By filling the blood vascular pixels using the surrounding non-vascular pixels, and then processing the reference image and the plurality of contrasted images respectively based on the segmentation algorithm, a proportion of the blood vascular region present in the background region may be reduced. The accuracy of the first background region and the second background region obtained by subsequent segmentation is improved.

The optical flow algorithm refers to an algorithm used in the field of computer vision and image processing to characterize the movement of pixels in an image. For example, the optical flow algorithm may include a Lucas-Kanade (LK) algorithm, a Horn-Schunck (HS) algorithm, etc.

In some embodiments, the processing device may employ the optical flow algorithm to calculate movement between two frames of images using variations of image pixels in the time domain and correlations of movement between neighboring frames. For two frames of images Iᵢ, Iⱼ∈ [symbol] ^{m×n}, the optical flow algorithm may be utilized to calculate the movement Dᵢⱼ∈ [symbol] ^{m×n×2} between the two frames of the images, with each pixel value in Dᵢⱼ representing the movement between Iᵢ and Iⱼ along an x-direction or a y-direction of the movement. Descriptions regarding the x-direction and y-direction may be found in the corresponding description above. Exemplarily, the processing device may determine the movement Dᵢⱼ between the two frames of the image by equation (2):

$${D}_{i j} = {f}_{o} ( {I}_{i} , {I}_{j} ) , \qquad ( 2 )$$

where Dᵢⱼ denotes the movement between the two frames, Iᵢ, Iⱼ denote the images, and fₒ denotes the optical flow algorithm.

In some embodiments, the processing device may utilize the optical flow algorithm to calculate a variation in the time domain between a pixel point corresponding to each second vascular region and a pixel point corresponding to the first vascular region, to obtain a first vascular movements relationship D^{v}∈ [symbol] ^{k×N}^{ᵥ}^{×2}. Each pixel value in D^{v} denotes a movement along the x-direction or y-direction between the pixel point corresponding to the first vascular region and the pixel point corresponding to each second vascular region, k denotes a count of images of consecutive contrasted images on a multi-frame time sequence, and Nᵥ denotes the number of vascular pixels. Descriptions regarding the x-direction and y-direction may be found in the corresponding descriptions above.

In some embodiments, the processing device may utilize the optical flow algorithm to calculate a variation in the time domain between a pixel point corresponding to each of the second background region and a pixel point corresponding to the first background region, to obtain a first background movement relationship D^{nv}∈ [symbol] ^{k×N}^{ₙᵥ}^{×2}. Each pixel value in D^{nv} denotes a movement along the x-direction or y-direction between the pixel point corresponding to the first background region and the pixel point corresponding to each second background region, k denotes a count of images of consecutive contrasted images on the multi-frame time sequence, and Nₙᵥ denotes the number of non-vascular pixels. Descriptions regarding the x-direction and the y-direction may be found in the corresponding descriptions above.

In some embodiments, the processing device may utilize the optical flow algorithm to calculate a variation in the time domain between an image feature point corresponding to each of the second vascular regions and an image feature point corresponding to the first vascular region to obtain the first vascular movement relationship. Moreover, the processing device may utilize the optical flow algorithm to calculate the variation in the time domain between the image feature point corresponding to each of second background regions and the image feature point corresponding to the first background region, to obtain the first background movements relationship. The image feature point is an image pixel point that may characterize an image feature. For example, the image feature point may include an edge point. The processing device may determine the image feature point using a feature point extraction model. The feature point extraction model refers to an edge feature extraction algorithm. For example, the edge feature extraction algorithm may include a Histogram of Oriented Gradients (HOG) algorithm, a Laplacian of Gaussian (LoG) algorithm, a Sobel Operator (Sobel) algorithm. As another example, an edge feature extraction algorithm may be a deep learning-based edge feature extraction algorithm, etc.

The optical flow algorithm enables the detection and tracking of subtle movement variations in the image, which makes it possible to obtain the first vascular movements and the first background movements with high precision.

In 320, structural parameters of the movement determination model are determined based on the plurality of first vascular movements and the plurality of first background movements. In some embodiments, step 320 may be performed by the second determination module 220.

The movement determination model refers to a model for predicting a vascular movement of a target image relative to a reference image. The vascular movement of the target image relative to the reference image may be referred to as a second vascular movement. The second vascular movement may be used to determine a vascular region in the target image.

The movement determination model may be configured to determine a movement relationship of the blood vessels of the target image relative to the reference image. The movement relationship may characterize real-time movement of the blood vessels due to the movement of the respiration and/or heartbeat. The movement determination model may characterize a motion matrix of a vascular region between at least two frames of the plurality of contrasted images.

The structural parameters refer to parameters that reflect the structure of the movement determination model. The structural parameters may satisfy the determination of the first vascular movement based on the first background movement and the movement determination model. For example, utilizing the movement determination model with the structural parameters, the plurality of first vascular movements may be determined based on the plurality of first background movements.

It should be noted that heartbeat and/or respiration causes movement of some of the background regions (e.g., a soft tissue region, a visceral region, etc.), which drives the movement of the vascular region. Thus, structural parameters of the movement determination model may be determined based on the plurality of first vascular movements and the plurality of first background movements.

In some embodiments, the processing device may determine the structural parameters of the movement determination model based on a plurality of first vascular movements, a plurality of first background movements, and a predetermined relational model.

The predetermined relational model refers to an empirically determined model. Because of a limited count of contrasted images of the same object and a limited count of first vascular movements, and a limited count of the first background movements, there is a limited count of training samples for determining the structural parameters, and thus the predetermined relational model may be a linear model.

In some embodiments, the processing device may estimate a structural parameter Ŵ of the movement determination model based on the first vascular movement D^{v}∈ [symbol] ^{k×N}^{ᵥ}^{×2}, the first background movement D^{nv}∈ [symbol] ^{k×N}^{ₙᵥ}^{×2}, and the predetermined relational model g. Since the first vascular movement D^{v}, the first background movement D^{nv}, and the predetermined relational model g satisfy g (D^{nv}; W)=W·D^{v}, the processing device may estimate the structural parameter Ŵ of the movement determination model by the least squares algorithm, i.e., the structural parameters Ŵ of the movement determination model are determined by equation (3):

$$\hat{W} = arg {{min}_{W} ( {\hat{D}}^{v} - g ( {D}^{n v} ; W ) )}^{2} , \qquad ( 3 )$$

where Ŵ denotes the structural parameter of the movement determination model, D^{v} denotes the first vascular movement, and D^{nv} denotes the first background movement.

In some embodiments, the processing device may also determine the structural parameters of the movement determination model through other algorithms (e.g., a gradient descent algorithm).

The structural parameters of the movement determination model may be determined quickly and accurately by the least squares algorithm.

In some embodiments, the processing device may also determine, through fitting, the structural parameters of the movement determination model based on the plurality of first vascular movements, the plurality of first background movements, and other predetermined relational models. For example, the other predetermined relational models may include a quadratic model, a Gaussian model, etc. The specific form of the other predetermined relational model is not restricted.

In 330, a predictive vascular region in the target image is determined based on the movement determination model. In some embodiments, step 330 may be performed by the third determination module 230.

The target image is a non-contrasted image of the same object. The non-contrasted image refers to an image in which some or all of the blood vessels are not visualized by a contrast agent.

In some embodiments, the target image may be an image obtained by scanning imaging after a contrast agent is injected into a scan subject and the contrast agent is disappeared or partially disappeared. In some embodiments, the target image may be an image obtained by scanning imaging when the scan subject is not injected with the contrast agent.

The contrasted image and the target image are two images of the same object taken from the same patient. The contrasted image and the target image include a vascular region, the contrasted image is a medical image in a developing stage of the contrast agent, and the target image is a medical image in a non-developed stage or a partially developed stage of the contrast agent. The developing stage is a stage in which all of the blood vessels within the object are developed through the contrast agent. The non-developed stage is a stage in which all of the blood vessels within the object are not developed through the contrast agent. The partially developed stage is a stage in which a part of the blood vessels are visualized by the contrast agent and the other part of the blood vessels are not visualized by the contrast agent. It should be noted that the contrasted image and the target image may include two-dimensional image data of any of human head blood vessels, neck arterial blood vessels, pulmonary arterial blood vessels, pulmonary venous blood vessels, group arterial blood vessels, upper extremity arterial blood vessels, or lower extremity arterial blood vessels, e.g., an extended reality (XR) image. In some embodiments, the contrasted image or the target image is a digital subtraction angiography (DSA) image taken by the DSA device.

In some embodiments, the contrasted image and the target image are obtained by scanning the object during a current treatment, the contrasted image is obtained by scanning the object after injecting the contrast agent, and the target image is obtained by scanning the object after the contrast agent has completely or partially disappeared. In other embodiments, the contrasted image may include a plurality of consecutive contrasted frames obtained by detecting the object in a historical time period. In some embodiments, the target image is obtained by scanning the object during the current treatment, and the target image is obtained before injecting the contrast agent. In some embodiments, the target image is obtained by scanning the object during the current treatment, and the target image is obtained after the contrast agent is completely or partially disappeared.

The predictive vascular region refers to the vascular region in the target image.

In some embodiments, a vascular tracking process may further include determining whether the target image is a completely undeveloped image. The completely undeveloped image is an image of the object obtained after the contrast agent is completely disappeared or an image of the object obtained before the contrast agent is injected. For example, the processing device may determine whether a vascular image is displayed in the target image. When the vascular image is not displayed in the target image, the processing device may determine the target image as a completely undeveloped image. When the target image display at least a portion of the vascular image, the processing device may determine that the target image is not the completely undeveloped image. In some embodiments, in response to determining that the target image is the completely undeveloped image (e.g., without injecting the contrast agent, all of the contrast agent is disappeared), the processing device may calculate, based on the first background region and the target image (the entire target image is equivalent to the background region due to the blood vessels being non-developed), a movement of the background region (referred to as the second background movement) between the reference image and the target image utilizing the optical flow algorithm. Next, the processing device may derive, in conjunction with a pre-established movement determination model, a movement of the vascular region (referred to as the first vascular movement) between the reference image and the target image. Further, the processing device may determine the predictive vascular region in the target image based on the second vascular movement and the vascular region in the reference image (i.e., the first vascular region).

In some embodiments, in response to determining that the target image is the completely undeveloped image, for the target image Iⱼ, the processing device may calculate the second background movement

$${D}_{i j}^{n v}$$

between the target image and the reference image based on the first background region and the target image using the optical flow algorithm. Then the processing device may predict the second vascular movements

$${\hat{D}}_{i j}^{v}$$

between the reference image and the target image based on the structural parameter Ŵ of the movement determination model. For example, the second vascular movements is determined by equation (4):

$${\hat{D}}_{i j}^{v} = g ( {D}_{i j}^{n v} ; \hat{W} ) , \qquad ( 4 )$$

where

$${\hat{D}}_{i j}^{v}$$

denotes the second vascular movements,

$${D}_{ij}^{nv}$$

denotes the second background movement, and W denotes the structural parameter of the movement determination model.

In some embodiments, the processing device may also obtain the second background movement through other algorithms (e.g., a frame difference algorithm and a background difference algorithm).

In some embodiments, the vascular tracking process may further include determining whether the target image is an image of the same object. For example, the processing device may determine whether a background region (e.g., a bone region) of the target image is consistent with a background region (e.g., a bone region) of the reference image. When the background region of the target image and the background region of the reference image arc consistent, the processing device may determine that the target image is the image of the same object as the reference image. When the background region of the target image and the background region of the reference image are inconsistent, the processing device may determine that the target image is not the image of the same object as the reference image.

In some embodiments, in response to determining that the target image is the image of the same object as the reference image, the vascular tracking process may further include determining whether the target image is a partially developed image. The partially developed image may be an image of the object obtained after the contrast agent is partially disappeared. For example, the processing device may determine whether the vascular image is displayed in the target image and the vascular image is incomplete. When the vascular image is displayed in the target image and the vascular image is incomplete, the processing device may determine that the target image is the partially developed image. When the vascular image is not displayed in the target image or the vascular image is displayed in the target image and the vascular image is complete, the processing device may determine that the target image is not the partially developed image. The vascular image being complete means that the vascular image is continuous and extends to an edge of the image.

In some embodiments, in response to determining that the target image is a partial contrasted image of the same object (e.g., the contrast agent is partially disappeared), the processing device may extract the background of the target image based on the target image; and determine the predictive vascular region based on the background of the target image.

In some embodiments, in response to determining that the target image is the partially developed image of the same object, the processing device may process the target image separately based on the segmentation algorithm to determine a third background region of the target image. Next, the processing device may calculate a movement of a background region between the reference image and the target image as the second background movement based on the first background region and the third background region using the optical flow algorithm. Then, the processing device may derive, in combination with the pre-established movement determination model, the movement of the vascular region between the reference image and the target image as the second vascular movement. Further, the processing device may determine the predictive vascular region in the target image based on the second vascular movement and the vascular region (e.g., the first vascular region) in the reference image.

Exemplarily, in response to determining that the target image is the partially developed image, for the target image Iⱼ, the processing device may calculate the second background movement

$${D}_{i j}^{n v}$$

between the target image and the reference image based on the first background region and the third background region using the optical flow algorithm. The processing device may then predict the second vascular movement between the reference image and the target image based on the structural parameter Ŵ of the movement determination model by equation (4).

In some embodiments, the processing device may construct a first predetermined database to determine the predictive vascular region based on the first predetermined database and the background of the target image. The processing device may record historical background images and their corresponding historical vascular regions from historical data of all scan subjects into the database to form the first predetermined database. In some embodiments, the processing device may perform an image similarity calculation between the background of the target image and the historical background image in the first predetermined database. The processing device may select a historical blood vascular region corresponding to the historical background image with the highest similarity as the predictive vascular region. The similarity may be calculated by a mean squared error (MSE) algorithm, a structural similarity (SSIM) algorithm, a peak signal-to-noise ratio (PSNR) algorithm, etc.

In some embodiments, the processing device may extract, by a feature point extraction model, at least one image feature point of the background of the target image based on the background of the target image. The processing device may determine the predictive vascular region based on the at least one image feature point of the background of the target image. Descriptions regarding the feature point extraction model may be found in the corresponding description above.

In some embodiments, the processing device may construct a second predetermined database to determine the predictive vascular region based on the second predetermined database and the at least one image feature point of the background of the target image. The processing device may extract the at least one image feature point of the above historical background image from the historical background image in the historical data through the feature point extraction model, and record the at least one image feature point and its corresponding historical vascular region into the database to form the second predetermined database. For example, the processing device may construct a first vector using the at least one image feature point of the background of the target image, and construct a second vector using the at least one image feature point of the historical background image. The processing device may calculate the similarity between the first vector and the second vector, and select the historical blood vascular region corresponding to a second vector with the greatest similarity as the predictive vascular region. The similarity may be characterized by a vector distance. The vector distance may include a Euclidean distance, a Cosine similarity, a Manhattan distance, etc.

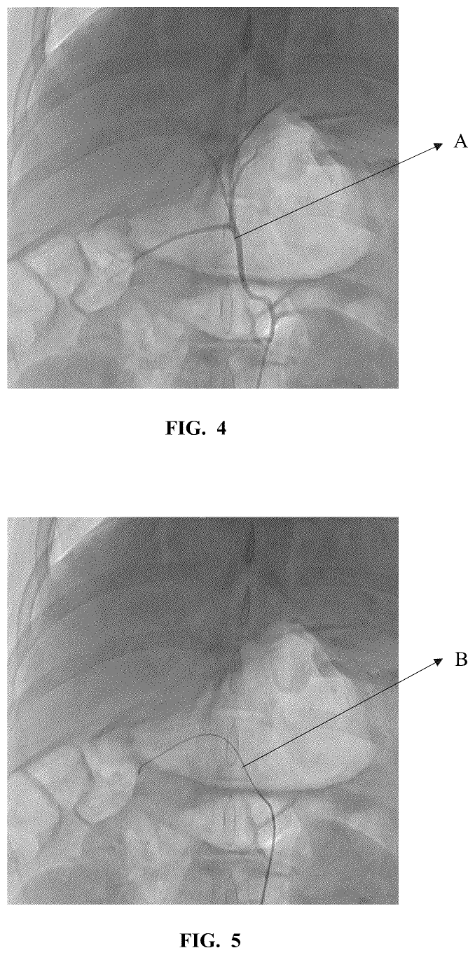

FIG. 4 is a schematic diagram illustrating a contrasted image according to some embodiments of the present disclosure. FIG. 5 is a schematic diagram illustrating a target image according to some embodiments of the present disclosure.

In some embodiments, as shown in FIG. 4, the region A indicates the vascular region of the contrasted image, and other regions indicate a background region of the contrasted image. The vascular region and the background region of the contrasted image are extracted by segmenting the contrasted image, and the vascular region and the background region are utilized to build a movement determination model. As shown in FIG. 5, the region B indicates a predictive vascular region. By calculating a second background movement between the target image and the reference image, and then utilizing the movement determination model to predict the second vascular movement based on the second background movement. A vascular movement in the target image after the disappearance of a contrast agent in each frame is tracked in real time to determine a specific location of the predictive vascular region.

In some embodiments of the present disclosure determining the structural parameters of the movement determination model based on the plurality of contrasted images, and then determining the predictive vascular region in the target image based on the movement determination model may accurately determine the predictive vascular region in the target image without any temporal phase information. The temporal phase information refers to variation information of imaging results obtained by imaging the same object for a plurality of times in medical imaging. The imaging results are observed over time.

In order to further improve the accuracy of the prediction of the vascular region in the target image, time-domain filtering of the movement of the vascular points in different contrasted images may be performed on the movement of the vascular points to get reliable vascular points. The vascular points refer to pixel points on the vascular region. The time-domain filtering may include a Gaussian-based outlier filter, a moving average filter, a median filter, a Butterworth filter, etc.

In some embodiments, the first vascular movement (e.g., denoted as D^{v}∈ [symbol] ^{k×N}^{ᵥ}^{×2}) is the vascular movement between the reference image and each frame of the plurality of contrasted images. The processing device may obtain a confidence level by the time-domain filtering. In some embodiments, the confidence level is denoted as F^{v}∈ [symbol] ^{k×N}^{ᵥ}. F^{v} denotes a physical quantity (e.g., a reliability degree) that may reflect whether the movement of a certain vascular point on one of the plurality of contrasted images is accurate, and k denotes a count of consecutive contrasted images on a multi-frame time series. For example, the processing device may combine a confidence level of a movement of a certain vascular point on one of the plurality of contrasted images with an interquartile range (IQR) to realize the time-domain filtering. The confidence level refers to an indicator that measures the reliability degree and trustworthiness of the data or results. The processing device may determine F^{v} by equation (5):

$${F}^{v} = {f}_{temporal} ( {D}^{v} ) , \qquad ( 5 )$$

where F^{v} denotes the confidence level (e.g., the reliability degree) of the movement of a vascular point on one of the plurality of contrasted images, and D^{v} denotes the first vascular movement.

Understandably, equation (3) may be replaced by equation (6) when the movement of the vascular points is filtered in the time domain:

$$\hat{W} = arg {{min}_{W} ( {D}^{v} ⊗ {F}^{v} - g ( {D}^{n v} , {F}^{v} ; W ) )}^{2} , \qquad ( 6 )$$

where Ŵ denotes the structural parameters of the movement determination model, D^{v} denotes the first vascular movement, D^{nv} denotes the first background movement, and F^{v} denotes the reliability of the movement of the vascular point on one of the plurality of contrasted images.

FIG. 7 is a schematic diagram illustrating an exemplary method for vascular tracking according to some embodiments of the present disclosure.

Exemplarily, as shown in FIG. 7, FIG. 7 shows the pipeline of a proposed method for vascular tracking consisting of three modules: a sparse corners alignment module, a movement-related model module, and a Gaussian-based Outlier Filtering (GOF) module. The sparse corners alignment module calculates a movement flow between a reference image Iᵣ and live moving frames. The sparse corners alignment module splits the movement flow into a vascular movements flow (e.g., a first vascular movement) and a non-vascular movements flow (e.g., a first background movement) with a vascular mask Mᵣ extracted from the reference frame. The movement determination model builds a correlation between the vascular movements flow and the non-vascular movements flow. The GOF module filters outliers with the non-vascular movements flow.

The robustness of the vascular tracking algorithm may be improved by dealing with the problem of vascular optical flow tracking error due to the inconsistency of blood vessels in the contrasted image sequence generated by the flow of contrast agent through a time domain filtering operation.

In some embodiments, the processing device may determine the first vascular movement and the first background movement without processing the plurality of contrasted images based on a segmentation algorithm. For example, the processing device may only determine a first vascular region in a reference image by processing the reference image e.g., based on the segmentation algorithm. The processing device may determine a plurality of first vascular movements based on the first vascular region and the plurality of contrasted images e.g., using an optical flow algorithm. As another example, the processing device may determine a first background region in the reference image by processing the reference image e.g., based on the segmentation algorithm. The processing device may determine a plurality of first background movements based on the first background region and the plurality of contrasted images e.g., using the optical flow algorithm.

After obtaining the first vascular region and the first background region, whether each pixel point in the reference image belongs to the first vascular region or the first background region may be determined. In the process of determining the plurality of first vascular movements or the plurality of first background movements e.g., using the optical flow algorithm, there is a corresponding relationship between each pixel point in the reference image and each pixel point in each of the plurality of contrasted images. Therefore, although the plurality of contrasted images are not segmented, whether each pixel point in the plurality of contrasted images belongs to a second vascular region or a second background region may be determined. The process of determining the first vascular movement is similar to the process of determining the plurality of first vascular movements based on the first vascular region and a plurality of second vascular regions e.g., using the optical flow algorithm. The process of determining the first background movement is similar to the process of determining the plurality of first background movement based on the first background region and a plurality of second background regions e.g., using the optical flow algorithm, which may not be repeated here.

In some embodiments, in response to determining that the target image is a partial contrasted image of the object, the processing device may determine a predictive vascular region based on the target image and a vascular region determination model. The vascular region determination model may be a trained machine learning model. Descriptions regarding the partial contrasted image may be found in the corresponding descriptions above. In some embodiments, the vascular region determination model may be a deep learning neural network model. The deep learning neural network model may include Convolutional Neural Networks (CNNs), Deep Neural Networks (DNNs), Recurrent Neural Networks (RNNs), etc., or a combination thereof.

In some embodiments, the vascular region determination model may include a first movement feature extraction layer, a first vascular feature extraction layer, and a vascular region determining layer. The processing device may determine a first movement feature based on the target image and the first movement feature extraction layer. The processing device may determine a first vascular feature based on the target image and the first vascular feature extraction layer. The processing device may determine the predictive vascular region based on the first vascular feature, the first movement feature, and the vascular region determining layer. The first vascular feature refers to a feature that may reflect a feature of a blood vessel that is visualized in the image. The first movement feature refers to a feature that may reflect a real-time movement of a blood vessel in the image that is caused by the movement of respiration and/or heartbeat. For example, the first vascular feature may include a length of the blood vessel, a width of the blood vessel, a count of the blood vessel, etc. The first movement feature may include a speed of movement, a trajectory of movement, etc.

An input of the first movement feature extraction layer may be the target image, and an output of the first movement feature extraction layer may be the first movement feature. An input of the first vascular feature extraction layer may be the target image, and an output of the first vascular feature extraction layer may be the first vascular feature. An input of the vascular region determining layer may be the first movement feature output from the first movement feature extraction layer and the first vascular feature output from the first vascular feature extraction layer, and an output of the vascular region determining layer may be the predictive vascular region.

In some embodiments, the output of the first movement feature extraction layer and the output of the first vascular feature extraction layer may be used as the input of the vascular region determining layer. The first movement feature extraction layer, the first vascular feature extraction layer, and the vascular region determining layer may be obtained by joint training.

In some embodiments, the vascular region determination model may be obtained by training a plurality of training samples with labels. The training samples may include a sample target image from historical data, and a label corresponding to a training sample may be an actual blood vascular region. The actual vascular region may be manually labeled or obtained by steps 310-330.

Training of the vascular region determination model may include one or more iterations. Merely by way of example, in a current iteration, for each of the training samples, the processing device may utilize an intermediate model to determine a sample predictive vascular region for the training sample. If the current iteration is a first iteration, the intermediate model may be an initial vascular region determination model. If the current iteration is an iteration other than the first iteration, the intermediate model may be a model generated in a previous iteration. The processing device may further determine a value of a loss function based on a similarity between the sample predictive vascular region and the actual vascular region, and update the intermediate model based on the value of the loss function.

In some embodiments, parameters of the intermediate model may be iteratively updated based on the plurality of training samples so that the loss function of the intermediate model satisfies a predetermined condition. For example, the loss function converges, or the value of the loss function is less than a predetermined value. Model training is complete when the loss function meets a predetermined condition, and the trained intermediate model may be designated as the vascular region determination model.

It should be noted that a training process for the machine learning model described below is similar to the training process of the vascular region determination model and may not be repeated subsequently.

With the vascular region determination model, the predictive vascular region may be predicted quickly and accurately. The joint training may reduce the count of training samples and improve the training efficiency while solving the problem of difficult to obtain the labels in training the first movement feature extraction layer and the first vascular feature extraction layer respectively.

In some embodiments, a movement determination model is a machine learning model, the processing device may obtain a plurality of training samples and a label. The plurality of training samples may include the plurality of first background movements, and the label corresponding to the plurality of training samples may include the plurality of first vascular movements. The processing device may train an initial movement determination model based on the plurality of training samples and a plurality of labels. The processing device may obtain the structural parameters of the movement determination model until a trained movement determination model satisfies a predetermined condition. Then, the processing device may determine a second background movement based on the target image and the first background region through the optical flow algorithm.

In some embodiments, a type of the movement determination model is similar to a type of the above vascular region determination model, which is not repeated here. More descriptions regarding the plurality of first background movements, plurality of first vascular movements, the second background movement, and the second vascular movement may be found in related descriptions hereinabove.

In some embodiments, there is at least one target image extracted based on a target video, the processing device may determine at least one predictive vascular region of the at least one target image and the movement determination model. The movement determination model is a trained machine learning model.

In some embodiments, the processing device may determine, based on the target image and the feature point extraction model, at least one image feature point of the target image. The processing device may determine the first movement feature based on the at least one image feature point of the target image and the first movement feature extraction layer. The processing device may determine the first vascular feature based on the at least one image feature point of the target image and the first vascular feature extraction layer. In other words, the input of the first movement feature extraction layer and the input of the first vascular feature extraction layer is the at least one image feature point of the target image determined by the feature point extraction model. Descriptions regarding the feature point extraction model may be found in the corresponding description above. The training samples may include at least one image feature point of the sample target image in the historical data, and the labels corresponding to the training samples may be actual vascular regions.

In some embodiments, the processing device may determine, based on a target video and the first movement feature extraction layer, the first movement feature. The processing device may determine, based on the target video and the first vascular feature extraction layer, the first vascular feature. The processing device may determine, based on the first vascular feature, the first movement feature and the vascular region determining layer, the predictive vascular region. The target video refers to a video obtained by scanning the object after injecting a contrast agent into the object and the contrast agent is disappeared or partially disappeared, or the target video refers to a video obtained by scanning the object when the object is not injected with the contrast agent. The target video may include a plurality of target image frames.

In some embodiments, the input of the first movement feature extraction layer may be the target video, and the output of the first movement feature extraction layer may be the first movement feature. The input of the first vascular feature extraction layer may be the target video, and the output of the first vascular feature extraction layer may be the first vascular feature. The input of the vascular region determining layer may be the first movement feature output from the first movement feature extraction layer and the first vascular feature output from the first vascular feature extraction layer, and the output of the vascular region determining layer may be the predictive vascular region. The training samples may include sample target videos from historical data, and the labels corresponding to the training samples may be actual vascular regions of the sample target videos.

Determining the predictive vascular region based on the target video may take into account movement features and vascular features of neighboring frames, which makes the obtained predictive vascular region accurate.

In some embodiments, the input of the vascular region determination model may further include the reference image and the plurality of contrasted images, and the vascular region determination model may further include a second movement feature extraction layer and a second vascular feature extraction layer.

In some embodiments, the processing device may determine a second movement feature based on the reference image, the plurality of contrasted images, and the second movement feature extraction layer. The processing device may determine a second vascular feature based on the reference image, the plurality of contrasted images, and the second vascular feature extraction layer. The processing device may determine the predictive vascular region based on the first vascular feature, the first movement feature, the second movement feature, the second vascular feature, and the vascular region determining layer. The second movement feature is similar to the first movement feature, and the second vascular feature is similar to the first vascular feature, which is not described herein.

In some embodiments, the input of the first movement feature extraction layer may be the target video, and the output of the first movement feature extraction layer may be the first movement feature. The input of the first vascular feature extraction layer may be the target video, and the output of the first vascular feature extraction layer may be the first vascular feature. The input of the second movement feature extraction layer may be the reference image and the plurality of contrasted images, and the output of the second movement feature extraction layer may be the second movement feature. The input of the second vascular feature extraction layer may be the reference image and the plurality of contrasted images, and the output of the second vascular feature extraction layer may be the second vascular feature. The input of the vascular region determining layer may be the first movement feature extraction layer output from the first movement feature, the first vascular feature extraction layer output from the first vascular feature, the second vascular feature output from the second movement feature extraction layer, and the second vascular feature output from the second vascular feature extraction layer. The output of the vascular region determining layer may be the predictive vascular region.

The training samples may include sample target videos from the historical data, sample reference images, and a plurality of sample contrasted images, and the labels corresponding to the training samples may be actual vascular regions of the sample target videos.

By adding inputs of the reference image and the plurality of contrasted images, the vascular region determination model may be tolerant to noise and abnormalities, and the predictive vascular region predicted by the vascular region determination model may be accurate.

In some embodiments, the processing device may determine at least one image feature point of the target video based on the target video and the feature point extraction model. The processing device may determine at least one image feature point of the reference image based on the reference image and the feature point extraction model. The processing device may determine at least one image feature point of plurality of contrasted images based on the plurality of contrasted images and the feature point extraction model. The processing device may determine the first movement feature based on the at least one image feature point of the target video and the first movement feature extraction layer. The processing device may determine the first vascular feature based on the at least one image feature point of the target video and the first vascular feature extraction layer. The processing device may determine the second movement feature based on the at least one image feature point of the reference image, the at least one image feature point of the plurality of contrasted images, and the second movement feature extraction layer. The processing device may determine the second vascular feature based on the at least one image feature point of the reference image, the at least one image feature point of the plurality of contrasted images, and the second vascular feature extraction layer. In other words, the inputs of the first movement feature extraction layer and the first vascular feature extraction layer are at least one image feature point of the target video determined by the feature point extraction model. The input of the second movement feature extraction layer and the second blood vascular feature extraction layer are at least one image feature point of the reference image and at least one image feature point of the plurality of contrasted images determined by the feature point extraction model.

Descriptions regarding the feature point extraction model may be found in the corresponding description above. The training samples may include at least one image feature point of the sample target videos from historical data, at least one image feature point of the sample reference images, and at least one image feature point of the plurality of sample contrasted images, and the labels corresponding to the training samples may be actual vascular regions of the sample target videos.

In some embodiments, there are a plurality of target images, the processing device may also extract the plurality of target images to form an image sequence based on a target video. The processing device may determine the predictive vascular region based on the target image sequence.

The target image sequence refers to a sequence including a plurality of target images (also referred to as the plurality of target image frames). In some embodiments, the processing device may randomly extract the target image sequence based on the plurality of target images. In other embodiments, the processing device may extract a predetermined count of target images according to a predetermined frame interval to form the target image sequence. For example, the processing device may extract 1 image at every interval of 5 frames among all the frames of the plurality of target images as a target image. The predetermined frame interval refers to a predetermined count of intervening frames between two consecutive target images to be extracted.

In some embodiments, the processing device may evaluate each image of the target image sequence, select one image with the clearest background and/or one image with the greatest count of blood vessels. The processing device may determine the predictive vascular region based on the image. The image with the clearest background may be an image with the greatest weighted value of contrast and resolution of the background.

In some embodiments, the processing device may construct a third predetermined database and determine a predictive vascular region based on the third predetermined database and the target image sequence.

In some embodiments, the processing device may construct the third predetermined database based on historical images in the historical data of all scan subjects, and the corresponding historical vascular regions thereof. For example, for one of the historical images in the third predetermined database, the processing device may perform a similarity calculation between each frame in the target image sequence and a historical image to determine an average similarity between the historical image and the target image sequence. The historical vascular region corresponding to the historical image with the greatest average similarity is designated as the predictive vascular region. Descriptions regarding the similarity may be found in the corresponding descriptions above.

In some embodiments, the processing device may extract a plurality of image feature points in the target image sequence based on the target image sequence and the feature point extraction model. In turn, the processing device may determine the predictive vascular region based on the plurality of image feature points of the target image sequence. Description regarding the feature point extraction model may be found in the corresponding descriptions above.

In some embodiments, the processing device may construct a fourth predetermined database to determine the predictive vascular region based on the fourth predetermined database and the plurality of image feature points of the target image sequence.

In some embodiments, the processing device may take a historical image from the historical data, extract at least one image feature point of the historical image by the feature point extraction model, and record the at least one image feature point of the historical image and the corresponding historical vascular region thereof into a database to form a fourth predetermined database. For example, the processing device may construct a first vector using the at least one image feature point of the sequence of target image, and construct a second vector using the at least one image feature point of the historical image having the same count of frames as the target image sequence, perform the similarity calculation between the first vector and the second vector, and select the historical blood vascular region corresponding to the second vector with the greatest similarity as the predictive vascular region. Descriptions regarding the vector similarity may be described above in the corresponding description.

In some embodiments, the processing device may determine at least one predictive vascular region based on the target video and the movement determination model. In some embodiments, the movement determination model may be a machine learning model. Descriptions regarding the target video may be found in the corresponding description above.

In some embodiments, the input of the movement determination model may be the target video, and the output of the movement determination model may be at least one predictive vascular region. The training samples may include sample target videos from the historical data, and the labels corresponding to the training samples may be determined in the following manner. The processing device may extract a plurality of image frames based on the target videos and perform operations in steps 310-330 on each of the plurality of image frames to determine the predictive vascular region until predictive vascular regions of all the images in the plurality of image frames are obtained, and designated the predictive vascular regions as the labels.

In some embodiments, the movement determination model may be obtained after a training process based on a training data set, a validating process based on a validation set, and a testing process based on a test set. The training set, the testing set, and the validation set are datasets composed of target videos from the historical data. An amount of data in the training set, an amount of data in the testing set, and an amount of data in the validation set constitute a predetermined ratio, and there is no overlapped data in the training set, the testing set, the validation set. A sample statistical difference of the training samples of the training set is greater than a predetermined difference threshold. The predetermined difference threshold is related to an error variance of the prediction of the historical vascular region.

The predetermined ratio refers to a pre-set ratio of the amount of data in the training set, the testing set, and the validation set. For example, the predetermined ratio of the training set, the validation set, and the test set may be 8:1:1.

The overlapped data means that a same data exists in the training set, the testing set, and the validation set. No overlapped data in the training set, the test set, and the validation set means that there is no same data existed in the training set, the test set, and the validation set.

The sample statistical difference refers to a difference of the training samples of the training set. The sample statistical difference may be denoted by a numerical value, a letter, or the like. In some embodiments, the sample statistical difference is represented by the numerical value, and the greater the diversity of the training samples of the training set, the greater the sample statistical difference.

In some embodiments, the processing device may convert each training sample in the training set into a vector based on python, such that each training sample corresponds to a numeric vector. Then the processing device may calculate a vector distance between every two training samples in the training set to obtain a plurality of vector distances, and calculate a variance based on the plurality of vector distances. The greater the variance, the greater the sample statistical difference. Descriptions regarding the vector distance may be found in the corresponding description above.

The predetermined difference threshold refers to a predetermined criterion for determining the magnitude of the sample statistical difference. In some embodiments, the predetermined variance threshold correlates to an error variance of the prediction of the historical vascular region. The greater the error variance of the prediction of the historical vascular region, the greater the predetermined difference threshold. Understandably, the greater the error variance of the prediction of the historical vascular region, the more uncertain the vascular prediction result and the more potential influences from various aspects. Thus, the predetermined variance threshold may be adjusted upward to allow the movement determination model to learn from more widely distributed data samples to more accurately learn the prediction of at least one predictive vascular region.

In some embodiments of the present disclosure, the movement determination model is obtained after being trained based on the training data set, validated based on the validation set, and tested based on the test set. Robustness of the movement determination model is improved and overfitting is prevented. At the same time, adjusting a predetermined difference threshold based on the error variance of the historical vascular region may make the training samples of the movement determination model extensive, thereby making the prediction result of the movement determination model accurate.

In some embodiments, the processing device may determine at least one predictive vascular region based on the target video, the target image, and the movement determination model.

In some embodiments, the input of the movement determination model may include the target video and the target image, and the output of the movement determination model may include the at least one predictive vascular region. The training samples may include the sample target videos and the sample target images in the historical data, and the labels corresponding to the training samples may be determined in the following manner. The processing device may extract a plurality of image frames from the target videos, and perform operations in steps 310-330 on each of the plurality of image frames and the target images, to determine the predictive vascular region until predictive vascular regions of all the plurality of image frames and the target images are obtained, and designate the predictive vascular regions as the labels.

In some embodiments, the processing device may determine the structural parameters based on the plurality of first vascular movements, the plurality of first background movements, and a structural parameter prediction model. The structural parameter prediction model is a machine learning model. The type of the structural parameter prediction model is similar to the type of the movement determination model and is not repeated here.

In some embodiments, an input of the structural parameter prediction model may include the plurality of first vascular movements and the plurality of first background movements, and an output of the structural parameter prediction model may be the structural parameters. Training samples of the structural parameter prediction model may include a plurality of sample first vascular movements and a plurality of sample first background movements in the historical data. Labels corresponding to the training samples may be determined as follows. The processing device may train a reference predetermined model based on the plurality of sample vascular movements and the plurality of sample background movements in the historical data and actual vascular regions, and designate structural parameters of the reference predetermined model as the labels.

In some embodiments, the processing device may determine the structural parameters based on the first vascular region, the first background region, the plurality of second vascular regions, the plurality of second background regions, and the structural parameter prediction model.

In some embodiments, the input of the structural parameter prediction model may include the first vascular region, the first background region, the plurality of second vascular regions, and the plurality of second background regions. The output of the structural parameter prediction model may be the structural parameters. The training samples of the structural parameter prediction model may include sample first vascular regions, sample first background regions, a plurality of sample second vascular regions, and a plurality of sample second background regions in historical data. The labels corresponding to the training samples arc determined in a manner similar to the manner of determining the labels of the structural parameter prediction model described above and may not be repeated herein.

In some embodiments, the processing device may determine the structural parameters based on the first vascular region, the plurality of second vascular regions, the plurality of first background movements, and the structural parameter prediction model.

In some embodiments, the input of the structural parameter prediction model may include the first vascular region, the plurality of second vascular regions, and the plurality of first background movements. The output of the structural parameter prediction model may be the structural parameters. The training samples of the structural parameter prediction model may include the sample first vascular regions, the plurality of sample second vascular regions, and the plurality of sample first background movements in the reference images in historical data. The labels corresponding to the training samples are determined in a manner similar to the manner of determining the labels of the structural parameter prediction model described above and may not be repeated herein.

In some embodiments, the processing device may determine the structural parameters based on the first background region, the plurality of second background regions, the plurality of first vascular movements, and the structural parameter prediction model.

In some embodiments, the input of the structural parameter prediction model may further include the first background region, the plurality of second background regions, and the plurality of first vascular movements. The output of the structural parameter prediction model may be the structural parameters. The training samples of the embodiment may include the sample first background regions, the plurality of sample second background regions, and the plurality of sample first vascular movements in the historical data. The labels corresponding to the training samples are determined in a manner similar to the manner of determining the labels of the structural parameter prediction model described above and may not be repeated herein.

In some embodiments, the processing device may determine the structural parameters based on the first vascular region, the plurality of second vascular regions, the first background region, the plurality of second background regions, the plurality of first background movements, the plurality of first vascular movements, and the structural parameter prediction model.

In some embodiments, the input of the structural parameter prediction model may include the first vascular region, the plurality of second vascular regions, the first background region, the plurality of second background regions, the plurality of first background movements, and the plurality of first vascular movements. The output of the structural parameter prediction model may be the structural parameters. The training samples of the embodiment may include the sample first vascular regions, the plurality of sample second vascular regions, the sample first background regions, the plurality of sample second background regions, the plurality of sample first background movements, and the plurality of sample first vascular movements. The labels corresponding to the training samples are determined in a manner similar to the manner of determining the labels of the structural parameter prediction model described above and may not be repeated herein.

In some embodiments, the processing device may obtain a vascular composite movement feature based on the plurality of first vascular movements. The processing device may obtain a background composite movement feature based on the plurality of first background movements. The processing device may determine the structural parameters of the movement determination model based on the vascular composite movement feature, the background composite movement feature, and the structural parameters of the movement determination model.

The vascular composite movement feature refers to data that characterizes the plurality of first vascular movements. In some embodiments, the processing device may calculate at least one of a mean, a mode, a median, etc., of the data characterizing the plurality of first vascular movements as the vascular composite movement feature.

The background composite movement feature refers to data that characterize the plurality of first background movements. In some embodiments, the processing device may calculate at least one of a mean, a mode, a median, etc., of the data characterizing the plurality of first background movements as the background composite movement feature.

The training samples of the movement determination model may include a plurality of sample vascular composite movement features and a plurality of sample background composite movement features in the historical data. Labels corresponding to the training samples are determined in a manner similar to the manner of determining the labels of the structural parameter prediction model described above and may not be repeated herein.

In some embodiments, the processing device may determine the vascular composite movement feature based on the first vascular region, the plurality of second vascular regions, and a vascular composite movement feature model. The type of vascular composite movement feature model is similar to that of the vascular region determination model and is not described herein.

In some embodiments, an input of the vascular composite movement feature model may include the first vascular region and the plurality of second vascular regions. An output of the vascular composite movement feature model may include the vascular composite movement feature. Training samples of the vascular composite movement feature model may include the sample first vascular regions and the plurality of sample second vascular regions in the historical data. Labels corresponding to the training samples may be determined in the following manner. The processing device may determine the plurality of sample vascular movements based on the sample first vascular regions and the plurality of sample second vascular regions through step 310. Then, the processing device may calculate at least one of a mean, a mode, a median, etc., of the data of the plurality of sample vascular movements as the labels.

In some embodiments, the processing device may determine the vascular composite movement feature based on the first vascular region, the plurality of second vascular regions, the plurality of first vascular movements, and the vascular composite movement feature model.

In some embodiments, the input of the vascular composite movement feature model may include the first vascular region, a plurality of second vascular regions, and a plurality of first vascular movements, and the output of the vascular composite movement feature model may be the vascular composite movement feature. The training samples of the embodiment may include the sample first vascular movements, a plurality of sample second vascular regions, and a plurality of sample first vascular movements in historical data. The label corresponding to the training samples may be determined in the following manner. The processing device may determine the plurality of sample vascular movements based on the sample first vascular regions, the plurality of sample second vascular regions, through step 310, then the processing device may calculate at least one of the mean, the mode, the median, etc. of the data of the plurality of sample vascular movements and the sample first vascular movements as the label.

In some embodiments, the processing device may determine the background composite movement feature based on the first background region, the plurality of second background regions, and a background composite movement feature model. The type of background composite movement feature model is similar to that of the vascular region determination model and is not described herein.

In some embodiments, an input of the background composite movement feature model may include the first background region and the plurality of second background regions. An output of the background composite movement feature model may include the background composite movement feature. Training samples of the embodiment may include sample first background regions and a plurality of sample second background regions in the historical data. Label corresponding to the training samples may be determined in the following manner. The processing device may determine the plurality of sample second background regions based on the sample first background regions and the plurality of sample background movements by step 310. Then the processing device may calculate at least one of a mean, a mode, a median, etc., of data of the plurality of sample background movements as the labels.

In some embodiments, the processing device may determine the background composite movement feature based on the first background region, the plurality of second background regions, the plurality of first background movements, and the background composite movement feature model.

In some embodiments, the input of the background composite movement feature model may include the first background region, the plurality of second background regions, and the plurality of first background movements, and the output of the background composite movement feature model may include the background composite movement feature. The training samples of the background composite movement feature model may include the sample first background movements, the plurality of sample second background regions, and the plurality of sample first background movements in the historical data. The labels corresponding to the training samples may be determined in the following manner. The processing device may determine the plurality of sample background movements based on the sample first background region and the plurality of sample second background regions by step 310. Then, the processing device may calculate at least one of a mean, a mode, a median, etc., of data of the plurality of sample background movements and the sample first background movement as the labels.

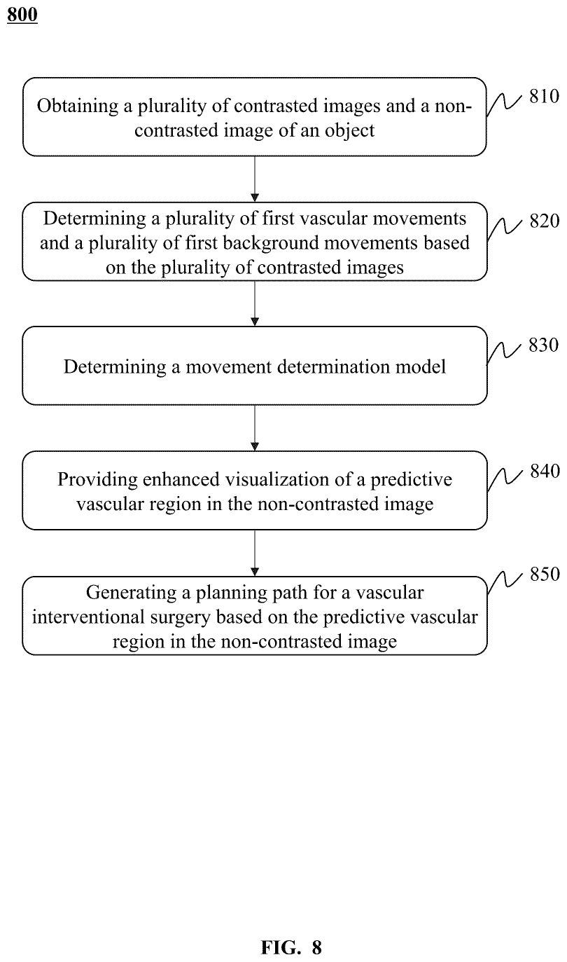

FIG. 8 is an exemplary flowchart illustrating an exemplary method for surgical path planning according to some embodiments of the present disclosure. As shown in FIG. 8, a process 800 includes the following operations.

In 810, a plurality of contrasted images and a non-contrasted image of an object are obtained. The non-contrasted image is an image of the object without any contrast agent. More descriptions regarding obtaining the plurality of contrasted images and the non-contrasted image of the object may be found in FIG. 2 and related descriptions thereof. In some embodiments, step 810 may be performed by a first acquiring module 910.

In 820, a plurality of first vascular movements and a plurality of first background movements are determined based on the plurality of contrasted images.

In 830, a movement determination model is determined.

In some embodiments, steps 820-830 are similar to steps 310-320, which is not repeated here. In some embodiments, steps 820-830 may be performed by the first determination module 210, and the second determination module 220, respectively.

In 840, enhanced visualization of a predictive vascular region in the non-contrasted image is provided. In some embodiments, step 840 may be performed by a providing module 920.

In 850, a planning path for a vascular interventional surgery is generated based on the predictive vascular region in the non-contrasted image. In some embodiments, step 850 may be performed by a generating module 930.

In some embodiments, a processing device may obtain the relative position of the surgical instrument in the vascular during an interventional surgery based on the positional information of the predictive vascular region. The positional information of the predictive vascular region may include position coordinates of the predictive vascular region in the target image. For example, the processing device may determine the relative position of the surgical instrument in the target image. In conjunction with the positional information of the predictive vascular region in the target image, the processing device may determine the relative position of the surgical instrument in the predictive vascular region. Combined with the preoperative path planned prior to the interventional surgery, the planning path for the vascular interventional surgery is obtained. For example, the processing device may generate a three-dimensional image of a blood vessel based on the relative position in the predictive vascular region by a seed-point algorithm or a region-growth algorithm. The processing device may determine a location of the pathology based on the three-dimensional image of the blood vessel. The region-growth algorithm refers to an iterative process that develops groups of pixels or regions into a larger region, maintaining a certain degree of similarity and connectivity during a merging process. The region-growth algorithm may be used to quickly, accurately, and reliably segment and recognize an object in an image. The planning path is generated based on the location of the pathology and a puncture navigation path required for the surgery within a three-dimensional spatial coordinate system. In such a case, it is possible to obtain a vascular path even after the disappearance of the contrast agent, which effectively assists the physician in the vascular interventional surgery by shortening the duration of the surgery, increasing the precision of the interventional surgery, and decreasing the amount of the contrast agent.

By predicting the positional information of the predictive vascular region and generating the planning path for the vascular interventional surgery, the problem of the inability to visualize the blood vessel for a long period of time due to the fast flow of the contrast agent is solved. The location of the blood vessel may be predicted in real time even after the disappearance of the contrast agent, which may effectively assist the doctor in the surgery. Thus, the time of interventional surgery is shortened, the accuracy of interventional surgery is improved, and the dose of the contrast agent is reduced.

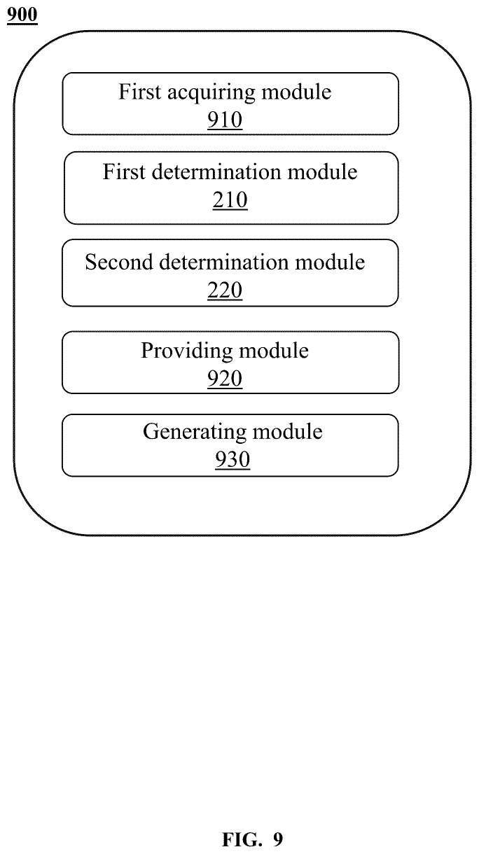

FIG. 9 is a diagram illustrating modules of an exemplary system for surgical path planning according to some embodiments of the present disclosure. In some embodiments, image 900 of the system for surgical path planning may include the first acquiring module 910, the first determination module 210, the second determination module 220, the third determination module 230, the providing module 920, and the generating module 930.

More descriptions regarding the first determination module 210 and the second determination module 220 may be found in FIG. 2 and related descriptions thereof.

The first acquiring module 910 may be configured to determine a movement determination model. The movement determination model may characterize a vascular movement of a vascular region between at least two frames of a plurality of contrasted images, a background movement of a background region between the at least two frames of the plurality of contrasted images, or a relationship between the vascular movement and the background movement.

The providing module 920 may be configured to provide enhanced visualization of a predictive vascular region in the non-contrasted image.

The generating module 930 may be configured to generate a planning path for a vascular interventional surgery based on the predictive vascular region in the non-contrasted image.

It is to be understood that the system and its modules shown in FIG. 9 may be realized using a plurality of approaches. It is to be noted that the above description of the system for surgical path planning and its modules are for descriptive convenience only and does not limit the present disclosure to the scope of the embodiments cited. It is to be understood that for a person skilled in the art, after understanding the principle of the system, it may be possible to arbitrarily combine the modules or form a sub-system to be connected to the other modules without deviating from this principle. In some embodiments, the first acquiring module 910, the first determination module 210, the second determination module 220, the providing module 920, and the generating module 930 disclosed in FIG. 9 may be different modules in a single system, or a single module realizing the aforementioned functions of two or more modules. For example, each module may share a common storage module, and each module may have a respective storage module. Such morphs are within the scope of protection of the present disclosure.

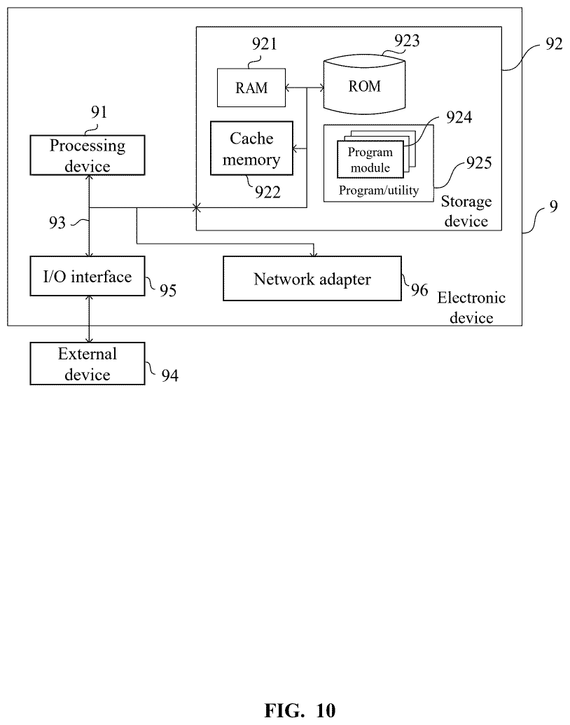

FIG. 10 is a schematic diagram illustrating an exemplary electronic device for vascular tracking in a medical image according to some embodiments of the present disclosure.

The electronic device may include a memory, a processing device, and a computer program stored on the memory and running on the processing device. The processing device executes the program to implement the method for vascular tracking or the method for surgical path planning. FIG. 10 shows that the electronic device 9 is merely an example and does not limit the functionality and scope of use of embodiments of the present disclosure.

As shown in FIG. 10, the electronic device 9 may be expressed in the form of a general-purpose computing device, for example, it may be a server device. Components of the electronic device 9 may include but are not limited to at least one processing device 91, at least one memory 92, a bus 93 connecting different system components including the memory 92 and the processing device 91.

The bus 93 consists of a data bus, an address bus, and a control bus.

The memory 92 may include a volatile memory, such as a random access memory (RAM) 921 and/or a cache memory 922, and may further include a read-only memory (ROM) 923.

The memory 92 may also include a program/utility 925 having a set (at least one) of program modules 924 such that the program modules 924 include but are not limited to an operating system, one or more applications, and other program modules and program data. Each of these examples, or some combination thereof, may include an implementation of a networked environment.

The processing device 91 performs various functional applications and data processing, such as the method for vascular tracking in the medical image or the method for surgical path planning of the embodiment of the present disclosure, by running the computer program stored in the memory 92.

The electronic device 9 may also in communication with one or more external devices 94 (e.g., keyboards, pointing devices, etc.). The communication may be implemented through an input/output (I/O) interface 95. In addition, the model-generated electronic device 9 may also in communication with one or more networks (e.g., a local area network (LAN), a wide area network (WAN), and/or a public network, such as the Internet) via a network adapter 96. The network adapter 96 is in communication with the other modules of the model-generated electronic device 9 via the bus 93, as shown in FIG. 10. It should be appreciated that, although not shown in the drawings, other hardware and/or software modules may be used in conjunction with the model-generated electronic device 9, including, but not limited to microcode, device drives, redundant processing devices, external disk drive arrays, disk array (RAID) systems, tape drives, and data backup storage systems, etc.

It should be noted that although referring to a plurality of units/modules or sub-units/modules of the electronic device in the detailed description above, such a division is merely exemplary and not mandatory. In fact, according to embodiments of the present disclosure, features and functions of two or more units/modules described above may be materialized in a single unit/module. Instead, features and functions of one unit/module described above may be further divided to be materialized by a plurality of units/modules.

The present embodiment provides a non-transitory computer readable storage medium having a computer program stored thereon, the program being executed by the processing device to realize the steps in the method for vascular tracking or the steps in the method for surgical path planning.

The readable storage medium may include but is not limited to a portable disk, a hard disk, a random access memory, a read-only memory, an erasable programmable read-only memory, an optical storage device, a magnetic storage device, or any suitable combination thereof.

In some embodiments, the present disclosure may also be realized in the form of a program product including program code that, when the program product is run on a terminal device, is configured to cause the terminal device to perform steps in the method for vascular tracking or steps in the method for surgical path planning.

The processing device may write program code for executing the present disclosure in any combination of one or more programming languages. The program code may be executed, entirely on a user device, partially on the user device, as a stand-alone software package, partially on the user device and partially on a remote device, or entirely on the remote device.

In some embodiments, a system for vascular tracking may include at least one storage medium including a set of instructions and at least one processor in communication with the at least one storage medium. When executing the set of instructions, the at least one processor is directed to cause the system to implement the method for vascular tracking.

In some embodiments, a system for surgical path planning may include at least one storage medium including a set of instructions and at least one processor in communication with the at least one storage medium. When executing the set of instructions, the at least one processor is directed to cause the system to implement the method for surgical path planning.

According to some embodiments of the present disclosure, a method for vascular tracking may be provided. A plurality of contrasted images of an object may be obtained. Each of the plurality of contrasted images may include a vascular region and a background region. A movement determination model may be determined based on a plurality of vascular regions and a plurality of background regions of the plurality of contrasted images. The movement determination model may characterize a vascular region movement (also referred to as a vascular motion) between different contrasted images, a background region movement (also referred to as a background motion) between different contrasted images, or a relationship between the vascular region movement and the background region movement. A predictive vascular region in a non-contrasted image of the object may be determined based on the movement determination model.

In some embodiments, a reference image may be obtained. A first vascular region in the reference image and a plurality of second vascular regions in the plurality of contrasted images may be determined. The plurality of first vascular movements may be determined based on the first vascular region and/or the plurality of second vascular regions. A first background region in the reference image and a plurality of second background regions in the plurality of contrasted images may be determined. The plurality of first background movements may be determined based on the first background region and the plurality of second background regions. More descriptions may be found elsewhere in the present disclosure, which are not repeated herein.

In some embodiments, the different contrasted images may be consecutive images in a time domain. The correlation between the different contrasted images may be extracted, thereby determining the movement determination model. In some embodiments, the correlation between the different contrasted images may be extracted by an algorithm (e.g., an optical flow algorithm). In some embodiments, the movement determination model may be determined based on artificial intelligence (AI). In such cases, the reference image may be unnecessary.

It should be noted the above descriptions are for illustration purposes. In some embodiments, a trained model may be obtained. The plurality of contrasted images may be input into the trained model and the movement determination model may be output by the trained model. In some embodiments, a trained model may be obtained. The plurality of contrasted images may be input into the trained model and the predictive vascular region in the non-contrasted image may be output by the trained model. In such cases, the reference image may be unnecessary.

In some embodiments, the system for vascular tracking may include a second acquiring module, a fourth determination module, and a fifth determination module.

The second acquiring module may be configured to obtain the plurality of contrasted images of the object, each of the plurality of contrasted images including a vascular region and a background region.

The fourth determination module may be configured to determine the movement determination model based on a plurality of vascular regions and background regions of the plurality of contrasted images. The movement determination model characterizes a vascular region movement between different contrasted images, a background region movement between different contrasted images, or a relationship between the vascular region movement and the background region movement.

The fifth determination module may be configured to determine the predictive vascular region in the non-contrasted image of the object based on the movement determination model. It should be noted that the above description of the system for vascular tracking and its modules is for descriptive convenience only and does not limit the present disclosure to the scope of the embodiments. It is to be understood that for a person skilled in the art, after understanding the principle of the system, it may be possible to arbitrarily combine each module or form a subsystem to connect with other modules without departing from this principle. In some embodiments, the second acquiring module, the fourth determination module, and the fifth determination module may be different modules in a system or may be a single module for implementing the functions of two or more of the above-described modules. For example, each module may share a common storage module, or may have a respective storage module. Such morphisms are within the scope of protection of the present disclosure.

According to some embodiments of the present disclosure, a method for vascular tracking may be provided. A movement determination model may be obtained. The movement determination model may represent a vascular region movement between different contrasted images, a background region movement between different contrasted images, or a relationship between the vascular region movement and the background region movement, of a prior object. A non-contrasted image of a latter object may be obtained. A predictive vascular region in the non-contrasted image of the object may be obtained based on the movement determination model. The prior object and the latter object may be the same or different.

According to some embodiments of the present disclosure, a method for vascular tracking may be provided. A non-contrasted image of an object may be obtained. A movement determination model may be obtained. The movement determination model may be able to predict a vascular region in a target image of the object without needing of the phase information of the object. A predictive vascular region in the non-contrasted image of the object may be determined based on the movement determination model.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment”, “an embodiment”, and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method for vascular tracking, comprising:
determining a plurality of first vascular movements and a plurality of first background movements of an object based on a reference image and a plurality of contrasted images of the object, wherein the reference image and the plurality of contrasted images are images of blood vessels within the object which provides enhanced visualization at a region of interest, the plurality of first vascular movements represent vascular movements in the plurality of contrasted images relative to the reference image, and the plurality of first background movements represent background movements in the plurality of contrasted images relative to the reference image;
determining a movement determination model based on the plurality of first vascular movements and the plurality of first background movements, wherein the movement determination model characterizes a vascular motion of a vascular region between at least two frames of the plurality of contrasted images, a background motion of a background region between the at least two frames of the plurality of contrasted images, or a relationship between the vascular motion and the background motion; and
determining a predictive vascular region in a target image based on the movement determination model, wherein the target image is a non-contrasted image of the object.

2. The method of claim 1, wherein the plurality of contrasted images include at least one of an X-ray image, a computed tomography (CT) image, or a magnetic resonance imaging (MRI) image.

3. The method of claim 1, wherein the determining the plurality of first vascular movements based on the reference image and the plurality of contrasted images of the object comprises:
determining a first vascular region in the reference image and a plurality of second vascular regions in the plurality of contrasted images; and
determining the plurality of first vascular movements based on the first vascular region and the plurality of second vascular regions.

4. The method of claim 1, wherein the determining the plurality of first background movements based on the reference image and the plurality of contrasted images of the object comprises:
determining a first background region in the reference image and a plurality of second background regions in the plurality of contrasted images; and
determining the plurality of first background movements based on the first background region and the plurality of second background regions.

5. The method of claim 1, wherein the determining the plurality of first vascular movements based on the reference image and the plurality of contrasted images of the object comprises:
determining a first vascular region in the reference image by processing the reference image; and
determining the plurality of first vascular movements based on the first vascular region and the plurality of contrasted images.

6. The method of claim 1, wherein the determining the plurality of first background movements based on the reference image and the plurality of contrasted images of the object comprises:
determining a first background region in the reference image by processing the reference image; and
determining the plurality of first background movements based on the first background region and the plurality of contrasted images.

7. The method of claim 6, wherein the determining the movement determination model based on the plurality of first vascular movements and the plurality of first background movements comprises:
determining, based on the plurality of first vascular movements, the plurality of first background movements, and a predetermined relational model, structural parameters of the movement determination model.

8. The method of claim 1, wherein the determining the predictive vascular region in the target image based on the movement determination model comprises:
determining a second background movement based on the target image and a first background region of the reference image;
determining a second vascular movement based on the second background movement and the movement determination model; and
determining the predictive vascular region based on a first vascular region of the reference image and the second vascular movement.

9. The method of claim 1, wherein the movement determination model is a trained machine learning model, and the determining the movement determination model based on the plurality of first vascular movements and the plurality of first background movements comprises:
obtaining a plurality of training samples and a plurality of labels, wherein the plurality of training samples include the plurality of first background movements, and the plurality of labels corresponding to the plurality of training samples includes the plurality of first vascular movements;
training an initial movement determination model based on the plurality of training samples and the plurality of labels; and
obtaining structural parameters of the movement determination model until a trained movement determination model satisfies a predetermined condition.

10. The method of claim 9, wherein there are multiple target images, and the method further includes:
extracting the multiple target images to form an image sequence based on a target video; and
determining the predictive vascular region based on the image sequence.

11. The method of claim 10, wherein the determining the movement determination model based on the plurality of first vascular movements and the plurality of first background movements comprises:
obtaining a confidence level by performing a time-domain filtering on the plurality of first vascular movements, wherein the confidence level denotes a reliability degree of a movement of a vascular point in one of the plurality of contrasted images; and
determining structural parameters of the movement determination model based on the plurality of first vascular movements, the plurality of first background movements, and the confidence level.

12. The method of claim 11, wherein the determining the first background region in the reference image and the plurality of second background regions in the plurality of contrasted images based on the reference image and the plurality of contrasted images through the segmentation algorithm respectively comprises:
obtaining a second reference image and a plurality of second contrasted images, the second reference image being a processed reference image and each of the second contrasted images being a processed contrasted image; and
determining the first background region and the plurality of second background regions based on the second reference image and the plurality of second contrasted images.

13. The method of claim 12, wherein there is at least one target image extracted based on a target video, and the method further includes:
determining at least one predictive vascular region of the at least one target image and the movement determination model, the movement determination model being a trained machine learning model.

14. The method of claim 1, wherein the determining the movement determination model based on the plurality of first vascular movements and the plurality of first background movements comprises:
determining structural parameters of the movement determination model based on the plurality of first vascular movements, the plurality of first background movements, and a structural parameter prediction model, the structural parameter prediction model being a trained machine learning model.

15. The method of claim 14, wherein the determining the movement determination model based on the plurality of first vascular movements and the plurality of first background movements comprises:
obtaining a vascular composite movement feature based on the plurality of first vascular movements;
obtaining a background composite movement feature based on the plurality of first background movements; and
determining structural parameters of the movement determination model based on the vascular composite movement feature and background composite movement feature.

16. A method for surgical path planning, comprising:
obtaining a plurality of contrasted images and a non-contrasted image of an object;
determining a plurality of first vascular movements and a plurality of first background movements based on the plurality of contrasted images, wherein the plurality of contrasted images include a reference image, the plurality of first vascular movements represent vascular movements of the plurality of contrasted images relative to the reference image, and the plurality of first background movements represent background movements of the plurality of contrasted images relative to the reference image;
determining a movement determination model, wherein the movement determination model characterizes a vascular motion of a vascular region between at least two frames of the plurality of contrasted images, a background motion of a background region between the at least two frames of the plurality of contrasted images, or a relationship between the vascular motion and the background motion;
providing enhanced visualization of a predictive vascular region in the non-contrasted image; and
generating a planning path for a vascular interventional surgery based on the predictive vascular region in the non-contrasted image.

17. (canceled)

18. (canceled)

19. A method for vascular tracking, comprising:
obtaining a plurality of contrasted images of an object, each of the plurality of contrasted images including a vascular region and a background region;
determining a movement determination model based on a plurality of vascular regions and a plurality of background regions of the plurality of contrasted images, wherein the movement determination model characterizes a vascular region movement between different contrasted images, a background region movement between different contrasted images, or a relationship between the vascular region movement and the background region movement; and
determining a predictive vascular region in a non-contrasted image of the object based on the movement determination model.

20. (canceled)

21. (canceled)

22. (canceled)

23. The method of claim 16, wherein the movement determination model is a machine learning model.

24. The method of claim 16, wherein the plurality of contrasted images include at least one of an X-ray image, a computed tomography (CT) image, or a magnetic resonance imaging (MRI) image.

25. The method of claim 16, wherein the first vascular movement is represented as a matrix, wherein each element of the matrix represents each pixel in the vascular region of the contrasted image and a movement of the each pixel along an x-direction and/or a y-direction in the contrasted image with respect to corresponding pixel in the vascular region of the reference image.
